(19) United States
(12) Patent Application Publication
(10) Pub. No.: US 2023/0348954 A1
(43) Pub. Date: Nov. 2, 2023

(54) DEVICES AND METHODS FOR SAMPLE ANALYSIS

(71) Applicant: Roche Sequencing Solutions, Inc., Pleasanton, CA (US)

(72) Inventors: Vladimira Datinska, Nezdenice (CZ); Keynttisha Jefferson, Madison, WI (US); Pantea Gheibi, San Jose, CA (US); Jaeyoung Yang, Pleasanton, CA (US)

(21) Appl. No.: 17/907,362

(22) PCT Filed: Mar. 24, 2021
(86) PCT No.: PCT/EP2021/057573

§ 371 Date: Sep. 26, 2022

Related U.S. Application Data
(60) Provisional application No. 63/000,022, filed on Mar. 26, 2020.

(51) Int. Cl. C12Q 1/6806; G01N 27/447

(52) U.S. Cl. CPC C12Q 1/6806; G01N 27/44713; G01N 27/44739

(57) ABSTRACT

The present disclosure generally relates to devices and methods for effecting epitachophoresis. Epitachophoresis may be used to effect sample analysis, such as by selective separation, detection, extraction, and/or pre-concentration of target analytes such as, for example, DNA, RNA, and/or other biological molecules. Said target analytes may be collected following epitachophoresis and used for desired downstream applications and further analysis.

### FIELD OF THE ART

The present disclosure generally relates to the field of electrophoresis, and more particularly to sample analysis by selective separation, detection, extraction, isolation, purification, and/or (pre-) concentration of samples such as, for example, samples comprising nucleic acids, through devices and methods for epitachophoresis.

### BACKGROUND

The use of nucleic acid-based analytical techniques, such as, for example, DNA sequencing, RNA sequencing, and gene expression profiling, are not only prevalent in various research applications but are also rapidly becoming a part of many therapeutic regimens. For example, the determination of gene expression levels in tissues is of great importance for accurately diagnosing human disease and is increasingly used to determine a patient's course of treatment. In some instances, pharmacogenomic methods can identify patients likely to respond to a particular drug and can lead the way to new therapeutic approaches.

One important source for this type of information comes in the form of preserved biological specimens, such as, for example, tissues that have underwent a fixation process. In some instances, such fixation processes comprise chemical fixation by using crosslinking fixatives, e.g., aldehyde-based fixatives. For example, in many instances, formaldehyde-based solutions may be used to produce formalin-fixed, paraffin-embedded tissue (“FFPET”) samples. FFPET samples are routinely created from biopsy specimens taken from patients undergoing diagnostic and/or therapeutic regimens for a variety of different diseases. These samples are usually associated with the corresponding clinical records and often play an important role in diagnosis and determination of treatment modality. For example, tumor biopsy FFPET samples are often linked with cancer stage classification, patient survival, and treatment regimen, thereby providing a potential wealth of information that can be cross-referenced and correlated with, for instance, gene expression patterns. However, the poor quality and quantity of nucleic acids isolated from FFPET samples using current methodologies has led to their general underutilization. For example, RNA isolated from FFPET samples is often moderately to highly degraded and fragmented, a significant drawback to its use in diagnostic and/or therapeutic applications. In addition to being degraded and fragmented, chemical modification of RNA by formalin can restrict the binding of oligo-dT primers to the polyadenylic acid tail of RNA, which can impede the efficiency of processes comprising the use of reverse transcription.

Moreover, conventional techniques for the extraction, isolation and/or purification of nucleic acids from preserved samples such as chemically fixed samples subjected to crosslinking fixatives, e.g., aldehyde-based fixatives, is often time-consuming, yields low quality product, and/or involves the use of harsh chemicals, such as organic solvents, e.g., xylene. Furthermore, conventional techniques generally use column and/or bead-based steps, which are costly, labor-intensive, and not well suited to automation. Therefore, further development of devices and methods for analyzing samples such as preserved specimens, e.g., FFPET samples, is needed.

### BRIEF SUMMARY

The present disclosure generally relates to a method of isolating and/or purifying one or more nucleic acids from a sample comprising one or more nucleic acids, optionally wherein said sample comprises a chemically fixed sample, further optionally a Formalin-Fixed Paraffin-Embedded Tissue (“FFPET”) sample, wherein said method comprises: a. providing a device for effecting epitachophoresis (“ETP”); b. providing the sample comprising said one or more nucleic acids; c. performing one or more epitachophoresis runs by effecting ETP using said device to focus said one or more nucleic acids into one or more focused zones; d. collecting said one or more nucleic acids by collecting said one or more focused zones comprising said one or more nucleic acids; thereby obtaining one or more isolated and/or purified nucleic acids, optionally wherein said nucleic acids comprise DNA and/or RNA. In some embodiments, prior to step c., a sample solution may be prepared from said sample comprising one or more nucleic acids. In some embodiments, said sample may comprise any one or more of an FFPET sample; an FFPET curls sample; and/or a sample solution prepared from either of said samples. In some embodiments, the sample may comprise an FFPET sample and an FFPET sample solution is prepared from the FFPET sample. In some embodiments, the nucleic acids may comprise DNA and/or RNA. In some embodiments, the isolated and/or purified nucleic acids may comprise DNA and/or RNA. In some embodiments, the isolated and/or purified nucleic acids collected from any single ETP run may comprise both DNA and RNA, optionally wherein the DNA and RNA are simultaneously isolated/purified and collected. In some embodiments, the isolated and/or purified nucleic acids collected from any single ETP run substantially or entirely may comprise either DNA or RNA, i.e., the DNA and RNA are substantially or entirely separately (i.e., not simultaneously) isolated/purified and collected. In some embodiments, the isolated and/or purified nucleic acids collected from any single ETP run may comprise a mixture of DNA and RNA, and the collected mixture of DNA and RNA is subjected to downstream separation prior to use of the DNA and/or RNA in one or more IVD assays. In some embodiments, the quantity of nucleic acids isolated and/or purified may be greater as compared to the quantity of nucleic acids obtained using a column-based or bead-based protocol as measured by a fluorometer-based method. In some embodiments, the quality of nucleic acids isolated and/or purified may be higher as compared to the quality of nucleic acids obtained using a column-based or bead-based protocol as measured by a quality control qPCR-based method. In some embodiments, 1.25 times or more, 1.5 times or more, 1.75 times or more, 2.0 times or more, 2.25 times or more, 2.5 times or more, 2.75 times or more, 3 time or more, 4 times or more, 5 times or more, 10 times or more, 100 times or more, or 1000 times or more nucleic acids may be collected as compared to the quantity of nucleic acids obtained using a column-based or bead-based protocol. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of the one or more nucleic acids comprised in the original sample being isolated and/or purified and collected. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of the isolated and/or purified one or more nucleic acids such as measured by an analytical technique to determine the composition of isolated/purified sample comprising one or more nucleic acids. In some embodiments, the quality of said isolated and/or purified nucleic acids may be determined by quality control qPCR. In some embodiments, the isolated and/or purified nucleic acids may be of any desired size. In some embodiments, the isolated and/or purified nucleic acids may be be 5 nt or less, 10 nt or less, 20 nt or less, 30 nt or less, 50 nt or less, 100 nt or less, 1000 nt or less, 10,000 nt or less, 100,000 nt or less, 1,000,000 nt or less, or 1,000,000 nt or more in size. In some embodiments, the method may comprise one or more pre-treatment steps. In some embodiments, said pre-treatment steps may comprise lysing said chemically fixed sample and/or preparing a sample solution. In some embodiments, said chemically fixed sample may comprise an FFPET curl, and said sample solution comprises an FFPET sample solution which is prepared by lysing said FFPET curl. In some embodiments, trailing electrolyte (“TE”) buffer may be added after lysis of said FFPET curl.

Moreover, the present disclosure generally relates to a method of isolating and/or purifying one or more nucleic acids from a Formalin-Fixed Paraffin-Embedded Tissue (“FFPET”) sample, wherein said method comprises: a. providing a device for effecting epitachophoresis (ETP); b. providing an FFPET sample comprising nucleic acids; c. preparing an FFPET sample solution from said FFPET sample; d. performing one or more epitachophoresis runs by effecting ETP using said device to focus said one or more nucleic acids into one or more focused zones; and e. collecting said one or more nucleic acids by collecting said one or more focused zones comprising said one or more nucleic acid; thereby obtaining one or more isolated and/or purified nucleic acids.

Furthermore, the present disclosure generally relates to a method of isolating and/or purifying one or more nucleic acids from a Formalin-Fixed Paraffin-Embedded Tissue (“FFPET”) sample, wherein said nucleic acids comprise DNA and RNA, wherein said method comprises: a. providing a device for effecting epitachophoresis (ETP); b. providing an FFPET sample comprising nucleic acids, wherein said FFPET sample optionally comprises one or more FFPET curls; c. preparing an FFPET sample solution from said FFPET sample, wherein said FFPET sample solution is optionally prepared by lysing one or more FFPET curls and adding trailing electrolyte (“TE”) buffer; d. performing one or more epitachophoresis runs by effecting ETP using said device to focus said one or more nucleic acids into one or more focused zones; and e. collecting said one or more nucleic acids by collecting said one or more focused zones comprising said one or more nucleic acid; thereby obtaining one or more isolated and/or purified nucleic acids.

Moreover, the present disclosure generally relates to a method of isolating and/or purifying one or more nucleic acids from a Formalin-Fixed Paraffin-Embedded Tissue (“FFPET”) sample, wherein said nucleic acids comprise DNA and RNA, wherein said method comprises: a. providing a device for effecting epitachophoresis (ETP); b. providing an FFPET sample comprising nucleic acids, wherein said FFPET sample optionally comprises one or more FFPET curls; c. preparing an FFPET sample solution from said FFPET sample, wherein said FFPET sample solution is optionally prepared by lysing one or more FFPET curls and adding trailing electrolyte (“TE”) buffer; d. performing one or more epitachophoresis runs by effecting ETP using said device to focus said one or more nucleic acids into one or more focused zones; and e. collecting said one or more nucleic acids by collecting said one or more focused zones comprising said one or more nucleic acid; thereby obtaining one or more isolated and/or purified nucleic acids, further wherein the isolated and/or purified nucleic acids collected from any single ETP run comprise both DNA and RNA.

Furthermore, the present disclosure generally relates to a method of isolating and/or purifying one or more nucleic acids from a Formalin-Fixed Paraffin-Embedded Tissue (“FFPET”) sample, wherein said nucleic acids comprise DNA and RNA, wherein said method comprises: a. providing a device for effecting epitachophoresis (ETP); b. providing an FFPET sample comprising nucleic acids, wherein said FFPET sample optionally comprises one or more FFPET curls; c. preparing an FFPET sample solution from said FFPET sample, wherein said FFPET sample solution is optionally prepared by lysing one or more FFPET curls and adding trailing electrolyte (“TE”) buffer; d. performing one or more epitachophoresis runs by effecting ETP using said device to focus said one or more nucleic acids into one or more focused zones; and e. collecting said one or more nucleic acids by collecting said one or more focused zones comprising said one or more nucleic acid; further wherein the isolated and/or purified nucleic acids collected from any single ETP run substantially or entirely comprise either DNA or RNA, i.e., the DNA and RNA are substantially or entirely separately (i.e., not simultaneously) isolated/purified and collected.

Moreover, the present disclosure generally relates to a method of isolating and/or purifying one or more nucleic acids from a Formalin-Fixed Paraffin-Embedded Tissue (“FFPET”) sample, wherein said nucleic acids comprise DNA and RNA, wherein said method comprises: a. providing a device for effecting epitachophoresis (ETP); b. providing an FFPET sample comprising nucleic acids, wherein said FFPET sample optionally comprises one or more FFPET curls; c. preparing an FFPET sample solution from said FFPET sample, wherein said FFPET sample solution is optionally prepared by lysing one or more FFPET curls and adding trailing electrolyte (“TE”) buffer; d. performing one or more epitachophoresis runs by effecting ETP using said device to focus said one or more nucleic acids into one or more focused zones; and e. collecting said one or more nucleic acids by collecting said one or more focused zones comprising said one or more nucleic acid; wherein the isolated and/or purified nucleic acids collected from any single ETP run comprises a mixture of DNA and RNA, and the collected mixture of DNA and RNA is subjected to downstream separation prior to use of the DNA and/or RNA in one or more IVD assays. In some embodiments, said isolated and/or purified nucleic acids are subjected to one or more in vitro diagnostic (“IVD”) assays.

### BRIEF DESCRIPTION OF THE SEVERAL VIEWS OF THE DRAWING

FIG. 1 provides a schematic representation of an exemplary device for effecting epitachophoresis.

FIG. 2A provides a schematic representation of a top view of an exemplary device for effecting epitachophoresis. In FIG. 2A, numbers 1-7 refer to the following: 1. Outer circular electrode; 2. Terminating electrolyte reservoir; 3. Leading electrolyte, optionally contained within a gel or otherwise hydrodynamically separated from the terminating electrolyte; 4. Leading electrolyte electrode/collection reservoir; 5. Central electrode; 6. Electric power supply; and 7. Boundary between leading and terminating electrolytes with sample ions focused in between; and the symbols r and d are used to represent the leading electrolyte reservoir radius and distance migrated by the LE/TE boundary, respectively.

FIG. 2B provides a schematic representation of a side view of an exemplary device for effecting epitachophoresis. In FIG. 2B, numbers 1-8 refer to the following: 1. Outer circular electrode; 2. Terminating electrolyte reservoir; 3. Leading electrolyte, optionally contained within a gel or otherwise hydrodynamically separated from the terminating electrolyte; 4. Leading electrolyte electrode/collection reservoir; 5. Center electrode; 6. Electric power supply; 7. Boundary between leading and terminating electrolytes with sample ions focused in between; and 8. Bottom support; and the symbols r and d are used to represent the leading electrolyte reservoir radius and distance migrated by the LE/TE boundary, respectively.

FIG. 3 provides a schematic representation of an exemplary device for effecting epitachophoresis.

FIG. 4 provides a schematic representation of an exemplary device for effecting epitachophoresis. In FIG. 4, the numbers 1-10 refer to the following: 1. Outer circular electrode; 2. Terminating electrolyte reservoir; 3. Leading electrolyte, optionally contained within a gel or otherwise hydrodynamically separated from the terminating electrolyte; 4. Opening to leading electrolyte/collection reservoir; 5. Center electrode; 6. Electric power supply; 7. Boundary between leading and terminating electrolytes with sample ions focused in between; 8. Bottom support; 9. Tube connecting device to a leading electrolyte reservoir; 10. Leading electrolyte reservoir.

FIG. 5 provides a schematic representation of an exemplary device for effecting epitachophoresis wherein the sample is loaded in between loading the leading and terminating electrolytes.

FIG. 6A provides a schematic representation of a device for effecting epitachophoresis and is referred to for the equations described in Example 2.

FIG. 6B provides a graph representing the travelled distance d in cm vs. the relative velocity at the distance d when an exemplary device for epitachophoresis (FIG. 6A) is operated using constant current. For the example presented in FIG. 6B, a radius value of 5 and starting velocity value of 1 were used.

FIG. 6C provides a graph representing the travelled distance d in cm vs. the relative velocity at the distance d when an exemplary device for epitachophoresis (FIG. 6A) is operated using constant voltage. For the example presented in FIG. 6C, a radius value of 5 and starting velocity value of 1 were used.

FIG. 6D provides a graph representing the travelled distance d in cm vs. the relative velocity at the distance d when an exemplary device for epitachophoresis (FIG. 6A) is operated using constant power. For the example presented in FIG. 6D, a radius value of 5 and starting velocity value of 1 were used.

FIG. 7 provides an image of an epitachophoresis device that was used to concentrate a sample in accordance with Example 3.

FIG. 8A provides an image of an exemplary device for epitachophoresis that was used in accordance with Example 4.

FIG. 8B provides an image of an exemplary device for epitachophoresis that was used to focus a sample into a focused zone in accordance with Example 4.

FIG. 8C provides an image of an exemplary device for epitachophoresis that was used to focus a sample into a focused zone in accordance with Example 4.

FIG. 9A provides an image of an exemplary device for epitachophoresis that was used in accordance with Example 5.

FIG. 9B provides a schematic representation of an exemplary device for epitachophoresis that was used in accordance with Example 5. In FIG. 9B, the numbers refer to dimensions in millimeters.

FIG. 9C provides an image of an exemplary device for epitachophoresis that was used to focus a sample into a focused zone in accordance with Example 5.

FIG. 9D provides an image of an exemplary device for epitachophoresis that was used to focus a sample into a focused zone in accordance with Example 5.

FIG. 10 provides an image of an exemplary device for epitachophoresis that was used to focus a sample into a focused zone in accordance with Example 5.

FIG. 11 provides an image of an exemplary device for epitachophoresis that was used to separate and to focus two different samples into focused zones in accordance with Example 5.

FIG. 12 provides an image of an exemplary device for epitachophoresis in accordance with Example 6.

FIG. 13A provides an image of an exemplary epitachophoresis device in accordance with Example 7.

FIG. 13B provides a schematic of an exemplary epitachophoresis device in accordance with Example 7. “a” corresponds to the central collection well and “b” corresponds to the leading electrolyte reservoir.

FIG. 14A provides an image of an exemplary conductivity measurement probe for use in an epitachophoresis device in accordance with Example 7.

FIG. 14B provides an image showing a closer view of the conductivity measurement probe shown in FIG. 14A.

FIG. 15A provides an image of an exemplary epitachophoresis device with a conductivity probe in accordance with Example 7.

FIG. 15B provides a conductivity trace for a run of an exemplary epitachophoresis device in accordance with Example 7.

FIG. 16A provides images of an exemplary epitachophoresis device with conductivity detecting probes placed underneath the semipermeable membrane in accordance with Example 7.

FIG. 16B provides images of an exemplary bottom substrate incorporating two conductivity detecting probes connected through dedicated channels within the central pillar.

FIG. 17A provides an image of an exemplary epitachophoresis device, demonstrating the focusing of a fluorescein-labeled DNA ladder sample in accordance with Example 7.

FIG. 17B provides a trace showing the resistivity change of the LE/TE transition monitored by a surface conductivity cell for a run of an exemplary epitachophoresis device in accordance with Example 7.

FIG. 17C provides absorbance spectra of the original sample and the collected fraction for the DNA ladder sample before and after an epitachophoresis run in accordance with Example 7.

FIG. 17D provides electropherograms for the DNA ladder sample before and after an epitachophoresis run, as measured via Bioanalyzer separations in accordance with Example 7.

FIG. 18 provides voltage profiles for three independent ETP runs in accordance with Example 8.

FIG. 19A provides an image of an ETP device during an ETP run in accordance with Example 9.

FIG. 19B provides a fluorescence-based image of an ETP device taken during an ETP run in accordance with Example 9.

FIG. 19C provides an image of an ETP device taken during an ETP run in accordance with Example 9.

FIG. 19D provides an electropherogram for the focused and collected ETP sample analyzed in accordance with Example 9.

FIG. 20A provides images captured using an infrared-based thermal imaging camera during at ETP run in accordance with Example 10.

FIG. 20B provides a fluorescence-based image captured during an ETP run in accordance with Example 10.

FIG. 20C presents data related to the voltage change and temperature change over time during an ETP run in accordance with Example 10.

FIG. 21A provides images of the ETP experimental setup in accordance with Example 11

FIG. 21B provides images of the ETP experimental setup in accordance with Example 11.

FIG. 22A provides an image of an ETP run in which Brilliant Blue dye was used as a sample marker in accordance with Example 11

FIG. 22B provides an image of an ETP run in which Brilliant Blue dye was used as a sample marker in accordance with Example 11.

FIG. 23 provides data from ETP-based isolation/purification of dsDNA from FFPET samples in accordance with Example 12. The quantity of dsDNA obtained by ETP-based isolation/purification was compared to the quantity obtained using a Promega column-based method as described in Example 12.

FIG. 24 provides data from ETP-based isolation/purification of RNA from FFPET samples in accordance with Example 12. The quantity of RNA obtained by ETP-based isolation and purification was compared to the quantity obtained using a Promega column-based method as described in Example 12.

FIG. 25A presents data from size-based analysis of dsDNA isolated/purified by ETP-based isolation/purification as compared to four different control samples in accordance with Example 13.

FIG. 25B presents data from qPCR-based analysis of dsDNA quality isolated/purified by ETP-based isolation/purification as compared to the quality of dsDNA obtained by a Promega column-based method in accordance with Example 13.

FIG. 26A provides data from ETP-based isolation/purification of dsDNA from a FFPET samples in accordance with Example 14. The quantity of dsDNA obtained by ETP-based isolation/purification was compared to the quantity obtained using a KAPA beads-based method.

FIG. 26B provides data from ETP-based isolation/purification of RNA from an FFPET samples in accordance with Example 14. The quantity of RNA obtained by ETP-based isolation and purification was compared to the quantity obtained using a KAPA beads-based method as described in Example 14.

FIG. 27 provides data related to DNase I-based analysis of nucleic acids obtained by ETP-based isolation/purification of FFPET samples in accordance with Example 15.

FIG. 28 provides data related to size-based analysis of nucleic acids obtained by ETP-based isolation/purification of FFPET samples before and after DNase I-treatment in accordance with Example 15.

FIG. 29 provides data related to comparison of the state-of-the-art FFPET extraction kit and the ETP method as described in Example 16.

FIG. 30 provides sequencing data of the nucleic acid extracted in Example 16.

### DETAILED DESCRIPTION

### Definitions

As used herein the singular forms “a”, “an”, and “the” include plural referents unless the context clearly dictates otherwise. All technical and scientific terms used herein have the same meaning as commonly understood to one of ordinary skill in the art to which this invention belongs unless clearly indicated otherwise.

As used herein, the term “isotachophoresis” generally refers to the separation of charged particles by using an electric field to create boundaries or interfaces between materials (e.g., between the charged particles and other materials in a solution). ITP generally uses multiple electrolytes, where the electrophoretic mobilities of sample ions are less than that of a leading electrolyte (LE) and greater than that of a trailing electrolyte (TE) that are placed in a device for ITP. The leading electrolyte (LE) generally contains a relatively high mobility ion, and a trailing electrolyte (TE) generally contains a relatively low mobility ion. The TE and LE ions are chosen to have effective mobilities respectively lower and higher than target analyte ions of interest. That is, the effective mobility of analyte ions is higher than that of the TE and lower than that of the LE. These target analytes have the same sign of charge as the LE and TE ions (i.e., a co-ion). An applied electric field causes LE ions to move away from TE ions and TE ions to trail behind. A moving interface forms between the adjacent and contiguous TE and LE zones. This creates a region of electric field gradient (typically from the low electric field of the LE to the high electric field of the TE). Analyte ions in the TE overtake TE ions but cannot overtake LE ions and accumulate (“focus” or form a “focused zone”) at the interface between TE and LE. Alternately, target ions in the LE are overtaken by the LE ions; and also accumulate at interface. With judicious choice of LE and TE chemistry, ITP is fairly generally applicable, can be accomplished with samples initially dissolved in either or both the TE and LE electrolytes, and may not require very low electrical conductivity background electrolytes.

As used herein, the term “epitachophoresis” generally refers to methods of electrophoretic separation that are performed using a circular or spheroid and/or concentric device and/or circular and/or concentric electrode arrangement, such as by use of the circular/concentric and/or polygonal devices as described herein. Due to a circular/concentric or another polygonal arrangement that is used during epitachophoresis; unlike conventional isotachophoresis devices, the cross section area changes during migration of ions and zones, and the velocity of the zone movement is not constant in time due to the changing cross sectional area. Thus, an epitachophoretic arrangement does not strictly follow conventional isotachophoretic principles, wherein the zones migrate with constant velocities. Notwithstanding these significant differences as shown herein epitachophoresis can be used to efficiently separate and focus charged particles by using an electric field to create boundaries or interfaces between materials that may have different electrophoretic mobilities (e.g., between the charged particles and other materials in a solution). LE and TE, as described for use with ITP, can be used for epitachophoresis as well. In some embodiments, epitachophoresis may be effected using constant current, constant voltage, and/or constant power. In some embodiments, epitachophoresis may be effected using varying current, varying voltage, and/or varying power. In some embodiments, epitachophoresis may be effected within the context of devices and/or an arrangement of electrodes whose shape may be described in general as circular or spheroid, such that the basic principles of epitachophoresis may be accomplished as described herein. In some embodiments, epitachophoresis may be effected within the context of devices and/or an arrangement of electrodes whose shape may be described in general as polygons, such that the basic principles of epitachophoresis may be accomplished as described herein. In some embodiments, epitachophoresis may be effected by any non-linear, contiguous arrangement of electrodes, such as electrodes arranged in the shape of a circle and/or electrodes arranged in the shape of a polygon.

As used herein, the terms “in vitro diagnostic application (IVD application)”, “in vitro diagnostic method (IVD method)”, “in vitro diagnostic assay”, and the like, generally refer to any application and/or method and/or device that may evaluate a sample for a diagnostic and/or monitoring purposes, such as identifying a disease in a subject, optionally a human subject. In some embodiments, said sample may comprise nucleic acids and/or target nucleic acids from a subject and/or from a sample, optionally further wherein said nucleic acids originated from preserved samples, such as preserved tissue samples, e.g., FFPET samples. In some embodiments, an epitachophoresis device may be used as an in vitro diagnostic device. In some embodiments, a target analyte that has been concentrated/enriched/isolated/purified through epitachophoresis may be used in a downstream in vitro diagnostic assay. In some embodiments, an in vitro diagnostic assay may comprise nucleic acid sequencing, e.g., DNA sequencing, e.g., RNA sequencing. In some embodiments, and IVD assay may comprise gene expression profiling. In some embodiments, an in vitro diagnostic method may be, but is not limited to being, any one or more of the following: staining, immunohistochemical staining, flow cytometry, FACS, fluorescence-activated droplet sorting, image analysis, hybridization, DASH, molecular beacons, primer extension, microarrays, CISH, FISH, fiber FISH, quantitative FISH, flow FISH, comparative genomic hybridization, blotting, Western blotting, Southern blotting, Eastern blotting, Far-Western blotting, Southwestern blotting, Northwestern blotting, and Northern blotting, enzymatic assays, ELISA, ligand binding assays, immunoprecipitation, ChIP, ChIP-seq, ChIP-ChiP, radioimmunoassays, fluorescence polarization, FRET, surface plasmon resonance, filter binding assays, affinity chromatography, immunocytochemistry, gene expression profiling, DNA profiling with PCR, DNA microarrays, serial analysis of gene expression, real-time polymerase chain reaction, differential display PCR, RNA-seq, mass spectrometry, DNA methylation detection, acoustic energy, lipidomic-based analyses, quantification of immune cells, detection of cancer-associated markers, affinity purification of specific cell types, DNA sequencing, next-generation sequencing, detection of cancer-associated fusion proteins, and detection of chemotherapy resistance-associated markers.

As used herein, the terms “leading electrolyte” and “leading ion” generally refer to ions having a higher effective electrophoretic mobility as compared to that of the sample ion of interest and/or the trailing electrolyte as used during ITP and/or epitachophoresis. In some embodiments, leading electrolytes for use with cationic epitachophoresis may include, but are not limited to including, chloride, sulphate and/or formate, buffered to desired pH with a suitable base, such as, for example, histidine, TRIS, creatinine, and the like. In some embodiments, leading electrolytes for use with anionic epitachophoresis may include, but are not limited to including, potassium, ammonium and/or sodium with acetate or formate. In some embodiments, an increase of the concentration of the leading electrolyte may result in a proportional increase of the sample zone and a corresponding increase in electric current (power) for a given applied voltage. Typical concentrations generally may be in the 10-20 mM range; however, higher concentrations may also be used.

As used herein, the terms “trailing electrolyte”, “trailing ion”, “terminating electrolyte”, and “terminating ion” generally refer to ions having a lower effective electrophoretic mobility as compared to that of the sample ion of interest and/or the leading electrolyte as used during ITP and/or epitachophoresis. In some embodiments, trailing electrolytes for use with cationic epitachophoresis may include, but are not limited to including, MES, MOPS, acetate, glutamate and other anions of weak acids and low mobility anions. In some embodiments, trailing electrolytes for use with anionic epitachophoresis may include, but are not limited to including, reaction hydroxonium ion at the moving boundary as formed by any weak acid during epitachophoresis.

As used herein, the term “focused zone(s)” generally refers to a volume of solution that comprises a component that has been concentrated (“focused”) as a result of performing epitachophoresis. A focused zone may be collected or removed from a device, and said focused zone may comprise an enriched and/or concentrated amount of a desired sample, e.g., a target analyte, e.g., a target nucleic acid. In the epitachophoresis methods described herein the target analyte generally becomes focused in the center of the device, e.g., a circular or spheroid or other polygonal shaped device.

As used herein, the terms “band” and “ETP band” generally refer to a zone (e.g. focused zone) of ion, analyte, or sample that travels separately from other ions, analytes, or samples during electrophoretic (e.g., isotachophoretic, or epitachophoretic) migration. A focused zone within an epitachophoresis device may alternatively be referred to as an “ETP band”. In some embodiments, an ETP band may comprise one or more types of ions, analytes, and/or samples. In some instances, an ETP band may comprise a single type of analyte whose separation from other materials present in a sample is desired, e.g., separation of target nucleic acid from cellular debris. In some instances, an ETP band may contain more than one desired analyte, e.g., polypeptides or nucleic acids sequences highly similar in sequence, e.g., allelic variants. In some instances, the ETP band may comprise different analytes of similar size or electrophoretic mobility. In such instances, the more than one desired analyte may be separated by further ETP runs, e.g., under different conditions that promote separation of said more than one analytes, and/or said more than one analyte may be separated by other techniques known in the art for separation of analytes, such as those described herein. In some embodiments, an ETP band may be collected and optionally subject to further analysis after one or more ETP-based isolations/purifications and collections. In some embodiments, an ETP band may comprise one or more target analytes undergoing or that have undergone ETP-based isolation/purification and optionally collection, e.g., as a part of an ETP-run.

The term “target nucleic acid” as used herein is intended to mean any nucleic acid to be detected, measured, amplified, isolated, purified, and/or subject to further assays and analyses. A target nucleic acid may comprise any single and/or double-stranded nucleic acid. Target nucleic acids can exist as isolated nucleic acid fragments or be a part of a larger nucleic acid fragment. Target nucleic acids can be derived or isolated from essentially any source, such as cultured microorganisms, uncultured microorganisms, complex biological mixtures, samples including biological samples, tissues, sera, ancient or preserved tissues or samples, environmental isolates or the like. Further, target nucleic acids include or are derived from cDNA, RNA, genomic DNA, cloned genomic DNA, genomic DNA libraries, enzymatically fragmented DNA or RNA, chemically fragmented DNA or RNA, physically fragmented DNA or RNA, or the like. In some embodiments, a target nucleic acid may comprise a whole genome. In some embodiments, a target nucleic acid may comprise the entire nucleic acid content of a sample and/or biological sample, e.g., an FFPET sample. Target nucleic acids can come in a variety of different forms including, for example, simple or complex mixtures, or in substantially purified forms. For example, a target nucleic acid can be part of a sample that contains other components or can be the sole or major component of the sample. Also, a target nucleic acid can have either a known or unknown sequence.

The term “target microbe” as used herein is intended to mean any unicellular or multicellular microbe, found in blood, plasma, other body fluids, samples such as biological samples, and/or tissues, e.g., one associated with an infectious condition or disease. Examples thereof include bacteria, archaea, eukaryotes, viruses, yeasts, fungi, protozoan, amoeba, and/or parasites. Furthermore, the term “microbe” generally refers to the microbe that may cause a disease, whether the disease is referred to or the disease-causing microbe is referred to.

As used herein, the term “biomarker” or “biomarker of interest” refers to a biological molecule found in tissues, blood, plasma, urine, and/or other body fluids that is a sign of a normal or abnormal process, or of a condition or disease (such as cancer). A biomarker may be used to see how well the body responds to a treatment for a disease or condition. In the context of cancer, a biomarker refers to a biological substance that is indicative of the presence of cancer in the body. A biomarker may be a molecule secreted by a tumor or a specific response of the body to the presence of cancer. Genetic, epigenetic, proteomic, glycomic, and imaging biomarkers can be used for cancer diagnosis, prognosis, and epidemiology. Such biomarkers can be assayed in non-invasively collected biofluids like blood, serum, and/or urine. Such biomarkers to be assayed can include those derived from any type of tissue sample, such as, for example, a chemically fixed tissue sample. In some instances, tissue samples may include FFPET samples. Biomarkers may be useful as diagnostics (e.g., to identify early stage cancers) and/or prognostics (e.g., to forecast how aggressive a cancer is and/or predict how a subject will respond to a particular treatment and/or how likely a cancer is to recur).

The term “sample” as used herein includes a specimen or culture (e.g., microbiological cultures) that includes or is presumed to include nucleic acids and/or one or more target nucleic acids. The term “sample” is also meant to include biological, environmental, and chemical samples, as well as any sample whose analysis is desired. A sample may include a specimen of synthetic origin. A sample may include one or more microbes from any source from which one or more microbes may be derived. A sample may include, but is not limited, to whole blood, skin, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), tissue samples, chemically fixed samples, FFPET samples, biopsy sample, urine, feces, sputum, saliva, nasal mucous, prostate fluid, semen, lymphatic fluid, bile, organs, bone marrow, tears, sweat, breast milk, breast fluid, embryonic cells and fetal cells. In some instances, the sample may be a fresh or frozen tissue. In some instances, the sample may be a sample treated with a chemical fixative, e.g., an aldehyde-based fixative. In some instances, the sample may be a formalin-fixed paraffin embedded tissue (“FFPET”) sample. In some instances, the sample may be of in vitro cultures established from cells taken from an individual, from which nucleic acids may be isolated/purified.

The term “target analyte” as used herein is intended to mean any analyte to be detected, measured, separated, concentrated, isolated, purified, and/or subject to further assays and analyses. In some embodiments, said analyte may be, but is not limited to, any ion, molecular, nucleic acid, biomarker, cell or population of cells, e.g., desired cells, and the like, whose detection, measurement, separation, concentration, and/or use in further assays is desired. In some embodiments, a target analyte may be derived from any of the samples described herein.

For purposes of the present disclosure, it will be understood that when a given component such as a layer, region, liquid or substrate is referred to herein as being disposed or formed “on”, “in” or “at” another component, that given component can be directly on the other component or, alternatively, intervening components (e.g., one or more buffer layers, interlayers, electrodes or contacts) can also be present. It will be further understood that the terms “disposed on” and “formed on” are used interchangeably to describe how a given component is positioned or situated in relation to another component. Hence, the terms “disposed on” and “formed on” are not intended to introduce any limitations relating to particular methods of material transport, deposition, or fabrication.

The term “communicate” is used herein to indicate a structural, functional, mechanical, electrical, optical, thermal, or fluidic relation, or any combination thereof, between two or more components or elements. As such, the fact that one component is said to communicate with a second component is not intended to exclude the possibility that additional components may be present between, and/or operatively associated or engaged with, the first and the second component.

As used herein, a “subject” refers to a mammalian subject (such as a human, rodent, non-human primate, canine, bovine, ovine, equine, feline, etc.) to be treated and/or one from whom a sample is obtained.

“Detecting” a sample within the context of an epitachophoresis device, system or machine may comprise detecting its position at one, several, or many points throughout the device. Detection may generally occur by any one or more means that do not interfere with desired device, system, or machine function and with methods performed using said device, system, or machine. In some embodiments, detection encompasses any means of electrical detection, e.g., through the detection of conductivity, resistivity, voltage, current, and the like. Furthermore, in some embodiments, detection may comprise any one or more of the following: electrical detection, thermal detection, optical detection, spectroscopic detection, photochemical detection, biochemical detection, immunochemical detection, and/or chemical detection. In some embodiments, one or more target analytes may be detected during ETP-based isolation/purification and optionally collection of said one or more target analytes. Moreover, sample detection within the context of ETP devices and methods of ETP are further described in U.S. Ser. Nos. 62/585,219 and 62/744,984; and PCT nos. PCT/EP2018/081049 and PCT/EP2019/077714 which disclosures are hereby incorporated by reference in their entirety herein.

In a sample analysis device or system, the term “sample collection volume” refers to a volume of sample intended for collection, e.g., by a robotic liquid handler, during or following analysis. In a device for effecting epitachophoresis, or a system comprising such a device, the sample collection volume is the volume intended for collection that comprises sample during or following epitachophoresis. In some embodiments, the sample collection volume may be located in the central well of a device or system described herein. In some embodiments, the sample collection volume may be located anywhere that permits collection of the desired sample. In some embodiments, the sample collection volume may be anywhere between the sample loading area and the leading electrolyte electrode/collection reservoir. The sample collection volume may be comprised by any suitable area, container, well, or space of the device or system. In some embodiments, the sample collection volume is comprised by a well, membrane, compartment, vial, pipette, or the like. In some embodiments, the sample collection volume may be formed by the space within or between components of the device or system, e.g. the space between two gels or a hole in a gel.

As used herein, the terms “ETP device”, “device for effecting ETP”, “device for ETP”, and the like, are used interchangeably to refer to devices which can perform, or on which can be performed, ETP and/or methods comprising ETP.

As used herein, the term “ETP-based isolation/purification” generally refers to devices and methods comprising ETP, e.g., devices on which ETP may be effected, e.g., methods comprising effecting ETP, wherein ETP focuses one or more target analytes into one or more focused zones (e.g., one or more ETP bands), thereby isolating/purifying the one or more target analytes from other materials comprised by an initial sample. It is noted the terms “isolate” and “purify” are used interchangeably. Furthermore, ETP based isolation/purification generally allows for subsequent collection of the one or more focused zones (one or more ETP bands) comprising said one or more target analytes. The degree of isolation/purification of one or more target analytes effected by one or more ETP-based isolations/purifications may be any degree or amount of isolation/purification of one or more target analytes from other materials. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of said target analyte, e.g., as measured by an analytical technique to determine the composition of an ETP isolated/purified sample comprising one or more target analytes. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of a target analyte being recovered from the original sample. In some embodiments, one or more ETP-based isolations/purifications may be effected to isolate/purify one or more target analytes, e.g., one or more nucleic acids. For example, in some instances, ETP-based isolation/purification may be effected on a sample comprising one or more target analytes to focus the one or more target analytes into one focused zone (ETP band), which substantially separates the one or more target analytes from other materials comprised in the original sample. The sample may be collected following ETP isolation/purification, and the isolated/collected sample may be further subject to another ETP-based isolation/purification. Optionally, the second ETP-based isolation-purification may be of such conditions so as to, in instances of more than one target analyte, isolate each of one or more target analytes into separate focused zones, each of which could optionally collected individually, thereby separating target analytes from one another, if desired.

As used herein, the term “mixed sample” generally refers to a sample comprising material from more than one source.

As used herein, the term “chemically fixed sample” generally refers to the preservation of a sample, e.g., a tissue sample, through use of chemical agents as is well-known in the art. For example, chemically fixed samples can include those samples that have been treated with a crosslinking fixative, such as an aldehyde-based fixative. Aldehyde based fixatives include those well-known in the art, such as, for example, formaldehyde-based fixatives and glutaraldehyde-based fixatives. In some instances, a chemically fixed sample may comprise an FFPET sample.

As used herein, the term “FFPET sample solution” generally refers to an FFPET sample in solution form, such as, for example, a solution comprising a lysed FFPET sample, that could, for instance, be loaded into a device for effecting ETP to isolate/purify one or more nucleic acids comprised in the FFPET sample.

As used herein, the terms “sample pre-treatment”, “pre-treated sample”, and the like, generally refers to any procedures performed on said samples prior to loading the sample onto an ETP device. For example, sample pre-treatment may comprise preparing an FFPET sample solution from an FFPET sample, such as by lysing FFPET curls under appropriate conditions for FFPET curl lysis. Such methods are known in the art, and include, for example, lysis protocols used as a part of KAPA Express Extract methods. For example, a lysis buffer may be added to an FFPET curl, and the solution subsequently heated until the desired degree of lysis is achieved.

### Methods and Devices

As noted above, current approaches to extracting, isolating, and/or purifying nucleic acids from preserved specimens, such as, for example, chemically fixed samples, e.g., FFPET samples, have many disadvantages. The general procedure to isolate nucleic acids from FFPET may include; removal of paraffin (deparaffinization), lysis of the preserved sample (protease digestion), reversal of cross-links acquired during the fixation process, and purification of nucleic acids. These protocols are typically complex, labor intensive, and yield low quantities of low quality nucleic acids. For example, nucleic acids harvested from FFPET samples using current approaches are often of poor quality and low quantity. For instance, RNA isolated from FFPET samples is often moderately to highly degraded and fragmented, a significant drawback to its use in diagnostic and/or therapeutic applications. Moreover, conventional techniques for the extraction, isolation and/or purification of nucleic acids from samples subjected to chemical fixation, e.g., chemical fixation comprising the use of aldehyde-based fixatives, often involves the use of harsh chemicals, such as organic solvents, e.g., xylene. Conventional techniques also generally use column and/or bead-based steps which are costly, labor-intensive, and not well suited to automation. To solve such problems, the present disclosure generally describes devices and methods for sample analysis, e.g., analysis of FFPET samples, comprising one or more nucleic acids, wherein said devices and methods comprise effecting epitachophoresis to isolate and/or purify said nucleic acids from FFPET samples, wherein the isolated and/or purified nucleic acids optionally may be subjected to further downstream assays, such as in vitro diagnostic (“IVD”) assays.

Furthermore, the present devices and methods allow for whole genome and/or whole nucleic acid content extraction from a sample and/or biological sample, whereas such an extraction would be difficult when using conventional capillary or microfluidic based devices and methods, in particular ITP-based capillary or microfluidic devices and methods. Additionally, the highly efficient extraction of target nucleic acids obtained through the use of the devices and methods described herein is helpful for downstream in vitro diagnostic (IVD) methods, in which the amount of target nucleic acid, e.g., DNA and/or RNA, directly correlates with the sensitivity that may be achieved in said down-stream IVD assay. Sometimes, spin columns or magnetic glass particles that bind nucleic acids on their surface conventionally may be used in order to effect extraction of nucleic acids. As compared to these conventional approaches, the devices and methods described herein may confer any one or more of the following advantages: higher extraction yields (potentially loss-less) compared to column- or bead-based extraction methods; a simpler device setup compared to the larger footprint for benchtop instruments; potentially faster sample turn-around and high parallelizability as compared to other devices applied to similar uses; easy integration with other microfluidics-based systems for down-stream processing of extracted nucleic acids. In some embodiments, the nucleic acids obtained by ETP-based isolation/purification of a sample, e.g., an FFPET sample, may comprise the total nucleic acid content of said sample, e.g., both DNA and RNA from said sample. In some instances, methods comprising ETP-based isolation/purification may comprise simultaneous collection of nucleic acids comprising DNA and RNA, and the collected nucleic acids may be subjected to methods for separating the DNA and RNA for further downstream assays, e.g., separation of DNA and RNA by any means known in the art.

Moreover, the present disclosure generally relates to a method of isolating and/or purifying one or more nucleic acids from a sample, e.g., an chemically fixed sample, wherein said method comprises: a. providing a device for effecting epitachophoresis (ETP); b. providing a sample comprising said one or more nucleic acids; c. performing one or more epitachophoresis runs by effecting ETP using said device to focus said one or more nucleic acids into one or more focused zones, e.g., as one or more ETP bands; and d. collecting said one or more nucleic acids by collecting said one or more focused zones comprising said one or more nucleic acids; thereby obtaining one or more isolated and/or purified nucleic acids, optionally wherein said sample comprises an FFPET sample solution comprising said nucleic acids.

In some embodiments, the method of isolating and/or purifying one or more nucleic acids from a sample, e.g., a chemically fixed sample, comprises: a. providing a device for effecting epitachophoresis (ETP); b. providing an FFPET sample comprising nucleic acids; c. preparing an FFPET sample solution from said FFPET sample; d. performing one or more epitachophoresis runs by effecting ETP using said device to focus said one or more nucleic acids into one or more focused zones, e.g., as one or more ETP bands; and e. collecting said one or more nucleic acids by collecting said one or more focused zones comprising said one or more nucleic acids; thereby obtaining one or more isolated and/or purified nucleic acids. In some instances, the nucleic acids may comprise DNA and/or RNA.

In some instances, the sample for ETP-based sample analysis may comprise any type of formaldehyde cross-linked biological sample. In some embodiments, said samples may comprise tissue samples, wherein optionally the tissue samples comprise animal tissues. In some embodiments, the samples may comprise samples embedded in paraffin. For example, the samples can be formalin fixed paraffin embedded tissue (FFPET). In some embodiments, the samples may have been obtained from an animal (e.g., a human) and then stored in a formaldehyde-containing solution to stabilize the sample prior to analysis, thereby cross-linking the nucleic acids and/or protein in the sample.

In some embodiments, said method for ETP-based isolation and/purification of one or more nucleic acids may be automated, e.g., by using an automated ETP system. See, for instance, U.S. Ser. Nos. 62/585,219 and 62/744,984; and PCT nos. PCT/EP2018/081049 and PCT/EP2019/077714, which disclosures are hereby incorporated by reference in their entirety herein.

In some embodiments, an ETP device for use with the methods described herein may comprise an ETP device as described in U.S. Ser. Nos. 62/585,219 and 62/744,984; and PCT nos. PCT/EP2018/081049 and PCT/EP2019/077714, which disclosures are hereby incorporated by reference in their entirety herein.

In some embodiments, said method may comprise ETP-based isolation and/or purification of one or more nucleic acids from one or more FFPET samples, wherein said nucleic acids comprise DNA and/or RNA. In some instances, said method may result in isolating and/or purifying both DNA and RNA in one or more ETP bands and/or focused zones during a single ETP run. In some instances, following ETP-based isolation and/or purification, RNA and DNA may be separated from one another and the RNA and/or DNA may be subjected to further downstream assays, such as one or more IVD assays, sequencing, and/or gene expression profiling. In some embodiments, said FFPET samples may comprise FFPET curls which are subsequently treated to produce an FFPET sample solution which may optionally be loaded into an ETP device.

In some embodiments, a device and/or method for epitachophoresis may focus and allow for collection of a target analyte in any desired amount of time that allows for a desired focusing and collection to occur. In some embodiments, said method may comprise effecting ETP for 120 minutes or more, 120 minutes or less, 100 minutes or less, 80 minutes or less, 60 minutes or less, 50 minutes or less, or 40 minutes or less.

In some embodiments, the nucleic acids obtained by ETP-based isolation/purification of nucleic acids from FFPET samples may be of a higher yield and/or higher quality as compared to nucleic acids obtained from FFPET samples using conventional techniques, such as those described supra, e.g., bead-based and/or column based methods. In some embodiments, the nucleic acids obtained by ETP-based isolation/purification of nucleic acids from one or more FFPET samples may be of an equal or higher quality as measured by qPCR-based analysis, e.g., a Q score obtained from said qPCR-based analysis such as quality control (qc) qPCR. It is noted that Q scores range from 0 (low quality) to 1 (high quality), and higher quality samples are generally preferred for downstream IVD applications, such as sequencing-based applications. In some embodiments, 1.25 times or more, 1.5 times or more, 1.75 times or more, 2.0 times or more, 2.25 times or more, 2.5 times or more, 2.75 times or more, 3 time or more, 4 times or more, 5 times or more, 10 times or more, 100 times or more, or 1000 times or more nucleic acids may be obtained using said a method comprising ETP-based isolation/purification of nucleic acids from one or more FFPET samples as compared to a bead-based and/or column-based method. In some embodiments, the amount of isolated and/or purified nucleic acids obtained from the methods described herein may be any amount and may at least in part on the sample used. In some instances, the amount of isolated and/or purified nucleic acids may range anywhere from a nanogram or less to macrograms or more.

In some embodiments, in order to cause movement of the charged particles in the present methods and devices, within a convenient time frame, the electric field strength may be about 10 V to about 10 kV with electric powers ranging from about 1 mW to about 100 W. In some embodiments, the maximum electric power applied for the fastest analysis may depend on the electric resistivity of the sample and electrolyte solutions and the cooling capabilities of the materials that may be used for construction of the devices described herein.

In some embodiments, said ETP-based isolation and/or purification of one or more nucleic acids from one or more chemically fixed samples may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of the one or more nucleic acids comprised in the original sample being isolated and collected. In some embodiments, said method may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of said one or more nucleic acids isolated/purified, e.g., as measured by an analytical technique to determine the composition of an ETP isolated/purified sample comprising one or more nucleic acids. In some embodiments, one or more of the buffer concentrations, e.g., LE and/or TE buffer concentrations; percentage of a gel comprised in said ETP device, and/or the stoppage time of the ETP-based isolation and collection run may be varied and/or optimized to enhance separation of said one or more nucleic acids from other materials comprised in the sample.

In some embodiments, the one or more nucleic acids to be isolated/purified by ETP-based isolation/purification may be any desired size. In some embodiments, the nucleic acids may be 5 nt or less, 10 nt or less, 20 nt or less, 30 nt or less, 50 nt or less, 100 nt or less, 1000 nt or less, 10,000 nt or less, 100,000 nt or less, 1,000,000 nt or less, or 1,000,000 nt or more in size. In some embodiments, said method further may comprise detection of said one or more nucleic acids during and/or after said ETP-based isolation and/or purification, e.g., said detection comprises optical detection, in some instances, wherein said optical detection comprises detection of an intercalating dye and/or an optical label which binds to and/or is associated with said one or more nucleic acids. In some embodiments, said detection may comprise electrical detection, e.g., voltage monitoring. In some embodiments, detection may comprise monitoring the movement of a dye, e.g., Brilliant Blue, and adjusting any one or more ETP parameters, e.g., starting or stopping sample collection, based on the movement of said dye.

In some embodiments, the method of ETP-based isolation/purification of one or more nucleic acids, optionally from one or more FFPET samples, may be an automated method wherein the sample is automatically loaded into said device, and/or said one or more nucleic acids are automatically collected from said device. In some embodiments, the one or more isolated and/or purified nucleic acids may be subject to one or more further ETP runs to further isolate and/or purify said one or more nucleic acids.

In some embodiments, said method further may comprise use of an ETP upper marker, such as those discussed in U.S. Application Ser. No. 62/847,699, which is hereby incorporated by reference in its entirety herein. In some embodiments, the isolated and/or purified nucleic acids may be further subject to CAncer Personalized Profiling by Deep Sequencing (CAPP-Seq). In some embodiments, the isolated and/or purified nucleic acids may be assayed for one or more biomarkers. In some embodiments, the isolated and/or purified nucleic acids may be further evaluated in one or more assays for the identification of both CNVs and infectious agents. In some embodiments, the isolated and/or purified nucleic acids may be further evaluated by one or more methods which detect quantitative and qualitative tumor-specific alterations of the nucleic acids, such as DNA strand integrity, frequency of mutations, abnormalities of microsatellites, and methylation of genes. In some embodiments, the isolated and/or purified nucleic acids may be further evaluated by one or more methods in order to detect diagnostic, prognostic, and monitoring markers, e.g., in the sample from a cancer patient. In some embodiments, said method may be further combined with CNV detection to provide a method for assisting with clinical diagnosis, treatments, outcome prediction and progression monitoring in patients with or suspected of having a malignancy. In some embodiments, the isolated and/or purified nucleic acids may be further subjected to methods for detecting genetic characteristics in a sample, including copy number variations (CNVs), insertions, deletions, translocations, polymorphisms and mutations. In some embodiments, the concentration of any one or more of the one or more isolated and/or purified nucleic acids may be determined. In some embodiments, the concentration may be determined by molecular barcoding. In some embodiments, the isolated and/or purified nucleic acids may be further analyzed for Tumor-Derived SNVs. In some embodiments, the nucleic acids may comprise nucleic acids that originate from one or more cancerous cells.

Furthermore, the present disclosure generally relates to a method of identifying tumor-derived SNVs comprising (a) obtaining a sample from a subject suffering from a cancer or suspected of suffering from a cancer, optionally wherein the sample is an FFPET sample, e.g., FFPET sample solution; (b) performing ETP-based isolation and/or purification to isolate and/purify target nucleic acids, to obtain an isolated and/or purified sample; (c) conducting a sequencing reaction on the isolated and/or purified sample to produce sequencing information; (d) applying an algorithm to the sequencing information to produce a list of candidate tumor alleles based on the sequencing information from step (c), wherein a candidate tumor allele comprises a non-dominant base that is not a germline SNP; and (e) identifying tumor-derived SNVs based on the list of candidate tumor alleles. In some embodiments, the candidate tumor allele may comprise a genomic region comprising a candidate SNV.

Furthermore, the present disclosure generally relates to a method of identifying viral-derived nucleic acids comprising (a) obtaining a sample, e.g., a chemically fixed sample, from a subject suspected to have a virus infection or suspected of having been exposed to a virus; (b) performing ETP-based isolation and/or purification to isolate and/or purify target nucleic acids to obtain an isolated and/or purified sample; (c) conducting a sequencing reaction on the isolated and/or purified sample to produce sequencing information; and (d) determining based on the sequencing information whether the subject has been infected with one or more viruses.

Moreover, in further exemplary embodiments, devices for sample analysis as described herein may comprise dimensions that accommodate 1 μl or less, 1 μl or more, 10 μl or more, 100 μl or more, 1 mL or more, 4 mL, or more, 5 mL or more, 10 mL or more, or 15 mL or more of sample volume. In some embodiments, the concentration of the one or more isolated and/or purified nucleic acids may be measured. In some embodiments, the sample volume of the sample to be loaded into an ETP device for ETP-based isolation/purification may be 0.25 mL or less, 0.25 mL or more, 0.5 mL or more, 0.75 mL or more, 1.0 mL or more, 2.5 mL or more, 5.0 mL or more, 7.5 mL or more, 10.0 mL or more, 12.5 mL or more, or 15.0 mL or more.

In further exemplary embodiments, said device may be used to concentrate a target analyte, e.g., from about 2 fold or more to about 1000 fold or more. In some embodiments, said target analyte may comprise one or more nucleic acids. In further embodiments, said target analyte may comprise small inorganic and organic ions, peptides, proteins, polysaccharides, DNA, or microbes such as bacteria and/or viruses.

In some embodiments, said nucleic acids collected by ETP-based isolation/purification may be used for one or more downstream in vitro diagnostic applications. Furthermore, in some embodiments the ETP device for sample analysis may be connected on-line to other devices, such as, for example, capillary analyzers, chromatography, PCR devices, enzymatic reactors, and the like, and/or any other device that may be used to effect further sample analysis, e.g., a device associated with IVD applications. In some embodiments, the ETP device may be used in a workflow with nucleic acid sequencing library preparation. Moreover, in some embodiments, the ETP device may be used with liquid handling robots that may optionally be used to effect downstream analysis of a sample that may have been focused and/or collected from said device.

In some embodiments, the method may comprise ETP-based isolation and purification of one or more nucleic acids comprising DNA and/or RNA, wherein said nucleic acids are isolated/purified from one or more biological samples. Such samples include, but are not limited to fresh samples or cell/tissue aspirates, frozen sections, needle biopsies, cell cultures, fixed tissue samples, cell buttons, tissue microarrays, and the like. In some embodiments, the sample comprises fixed paraffin-embedded tissue (e.g., FFPET) samples. While histological samples are typically fixed with an aldehyde fixative, such as formalin (formaldehyde) and glutaraldehyde, the methods described herein may additionally be implemented with tissues fixed using other fixation techniques such as alcohol immersion, and the like. In some instances, samples may comprise biopsies and fine needle aspirates and archived samples (e.g. tissue microarrays), and the like. In some instances, the samples may comprise, but are not limited to, FFPET samples from human tissues, laboratory animal tissues, companion animal tissues, or livestock animal tissues. Thus, for example, the samples include tissue samples from humans including, but not limited to samples from healthy humans (e.g., healthy human tissue samples), samples from a diseased subject and/or diseased tissue, samples used for diagnostic and/or prognostic assays and the like. Suitable samples also include samples from non-human animals. FFPET samples from, for example, a non-human primate, such as a chimpanzee, gorilla, orangutan, gibbon, monkey, macaque, baboon, mangabey, colobus, langur, marmoset, lemur, a mouse, rat, rabbit, guinea pig, hamster, cat dog, ferret, fish, cow, pig, sheep, goat, horse, donkey, chicken, goose, duck, turkey, amphibian, or reptile can be used in the methods described herein.

Furthermore, in some embodiments, FFPET samples of any age can be used with the methods described herein including, but not limited to, FFPET samples that are fresh, less than one week old, less than two weeks old, less than one month old, less than two months old, less than three months old, less than six months old, less than 9 months old, less than one year old, at least one year old, at least two years old, at least three years old, at least four years old, at least five years old, at least six years old, at least seven years old, at least eight years old, at least nine years old, at least ten years old, at least fifteen years old, at least twenty years old, or older.

In some embodiments, the fixed embedded tissue samples (e.g., FFPET samples) comprise an area of diseased tissue, for example a tumor or other cancerous tissue. While such FFPET samples find utility in the methods described herein, FFPET samples that do not comprise an area of diseased tissue, for example FFPET samples from normal, untreated, placebo-treated, or healthy tissues, also can be used in the methods described herein. In some embodiments of the methods described herein, a desired diseased area or tissue, or an area containing a particular region, feature or structure within a particular tissue, is identified in a FFPET sample, or a section or sections thereof, prior to isolation of nucleic acids as described herein, in order to increase the percentage of nucleic acids obtained from the desired region. Such regions or areas can be identified using any method known to those of skill in the art, including, but not limited to, visual identification, staining, for example hematoxylin and eosin staining, immunohistochemical labeling, and the like. In any event, in some embodiments, the desired area of the tissue sample, or sections thereof, can be dissected, either by macrodissection or microdissection, to obtain the starting material for the ETP-based isolation/purification of a nucleic acid sample using the methods described herein.

In certain illustrative, but non-limiting embodiments, the sample comprises a diseased area or tissue comprising cells from a cancer. In some embodiments the cancer comprises a cancer selected from the group consisting of acute lymphoblastic leukemia (ALL), acute myeloid leukemia (AML), Adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, Burkitt lymphoma, carcinoid tumors (e.g., childhood, gastrointestinal), cardiac tumors, cervical cancer, chordoma, chronic lymphocytic leukemia (CLL), chronic myelogenous leukemia (CIVIL), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, Langerhans cell cancer, Hodgkin lymphoma, hypopharyngeal cancer, intraocular melanoma, islet cell tumors, pancreatic neuroendocrine tumors, Kaposi sarcoma, kidney cancer (e.g., renal cell, Wilm's tumor, and other kidney tumors), Langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sezary syndrome), Hodgkin, non-Hodgkin, primary central nervous system (CNS)), macroglobulinemia, Waldenstrom, male breast cancer, malignant fibrous histiocytoma of bone and osteosarcoma, melanoma (e.g., childhood, intraocular (eye)), Merkle cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhabdomyosarcoma, soft tissue, uterine), Sezary syndrome, skin cancer (e.g., melanoma, Merkle cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenstrom macroglobulinemia, Wilm's tumor, and the like.

The devices and methods illustratively disclosed herein suitably may be practiced in the absence of any element which is not specifically disclosed herein and/or any element specifically disclosed herein.

### EXAMPLES

### Example 1: Devices for Epitachophoresis

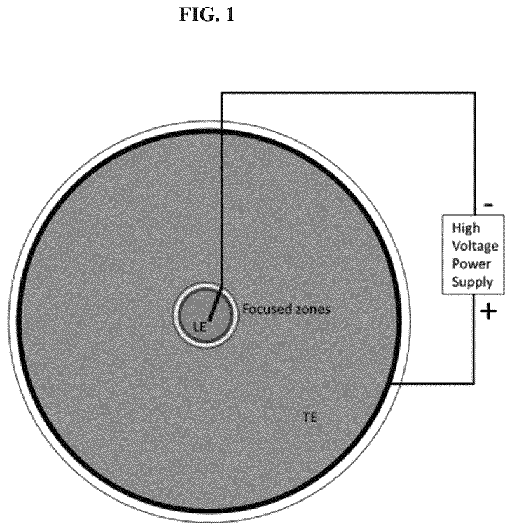

Devices for epitachophoresis generally use a concentric or polygonal disk architecture, for example, as depicted in FIG. 1-FIG. 4. Glass or ceramics are used for fabrication of the system (i.e. material for concentric or polygonal disks) as these materials result in improved heat transfer properties that are beneficial during device operation. For example, as the flat channel of a epitachophoresis device has a favorable heat transfer capability compared to a narrow channel, over-heating (or boiling) of the focused material is generally prevented. Current/voltage programming is also suitable for adjusting the Joule heating of the device. Plastic materials are also used for device fabrication. In general, devices are fabricated of such dimensions that accommodate a desired sample volume, such as milliliter-scale sample volumes, for example, up to 15 mL.

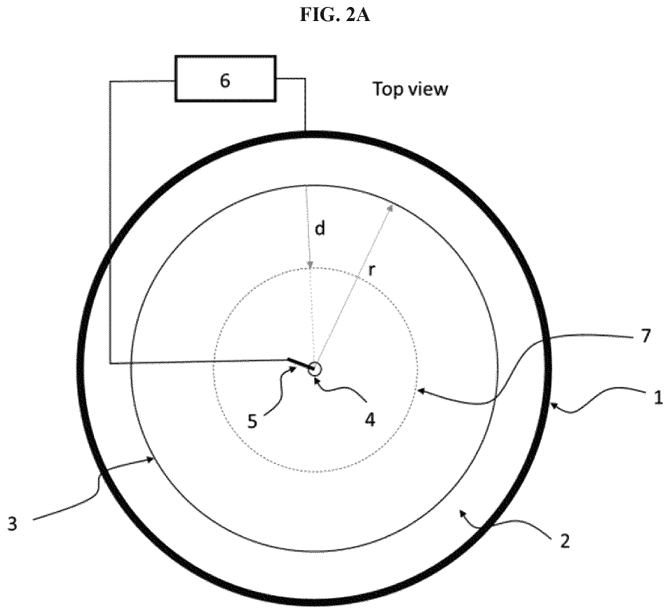

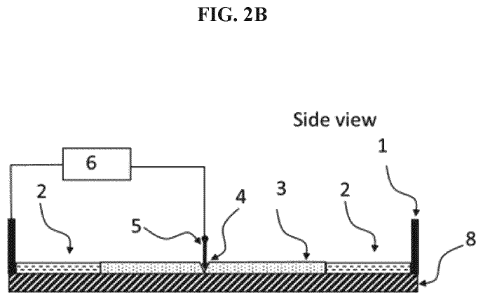

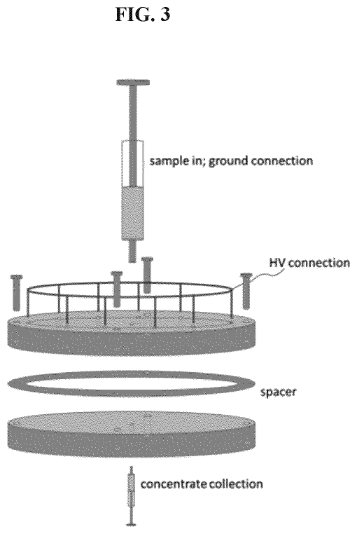

Referring to FIG. 1-FIG. 3, two concentric disks are separated by a spacer, thereby forming a flat channel for epitachophoresis sample processing. Electric current is applied through multiple high voltage connections (HV connection) and the ground connection in the center of the system (see FIG. 1 and FIG. 3, for example). In some instances, the sample is injected into the device through an opening in the device, e.g., in the top or the side (see, for example, FIG. 3). Application of electricity focuses the target analyte of a sample as a concentric ring that migrates to the center of the disk (discussed further below), and the target analyte is then collected through a syringe at the bottom of the device (see, for example, FIG. 3). As presented in FIG. 2A (top view) and FIG. 2B, an example of a device setup contains an outer circular electrode (1), terminating electrolyte (2), and leading electrolyte (3). In general, the diameter of the outer circular electrode (1) is about 10-200 mm and the diameter of the leading electrolyte ranges from a thickness (height) of about 10 μm to about 20 mm. The leading electrolyte is stabilized by a gel, viscous additive, or otherwise hydrodynamically separated from the terminating electrolyte, such as, for example, by a membrane. The gel or hydrodynamic separation prevents mixing of the leading and terminating electrolytes during device operation. Also, in some devices mixing is prevented by using very thin (<100 um) layers of electrolytes, as is discussed further below in Example 2.

Referring to FIG. 2A-FIG. 2B, in the center of the leading electrolyte is an electrode reservoir (4) with electrode (5). The assembly of the electrodes (1, 5) and electrolytes (2, 3) is placed on a flat, electrically insulating support (8). The electrolyte reservoir (4) is used for removal of the concentrated sample solution following a separation process, such as by pipetting the sample out of the reservoir, for example.

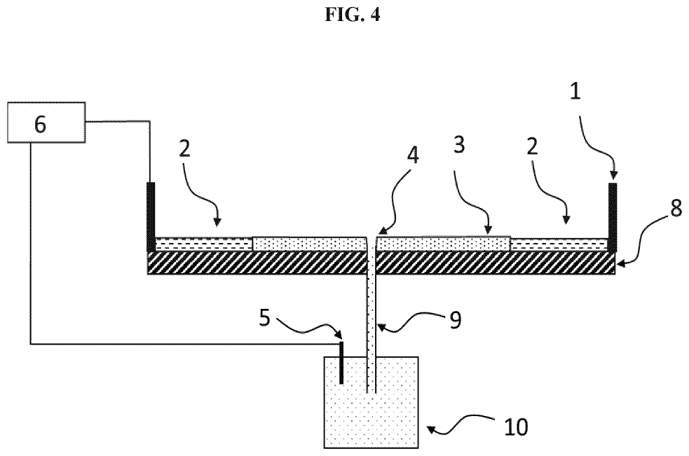

In an alternative arrangement (see FIG. 4) the center electrode (5) is moved to a leading electrolyte reservoir (10) connected with the concentrator by a tube (9). The tube (9) is connected directly or closed on one end by a semipermeable membrane (not shown). This arrangement facilitates the collection by stopping migration of large molecules according to the properties of the membrane used. This arrangement simplifies the sample collection and provides means of connecting the concentrator on-line to other devices, such as, for example, capillary analyzers, chromatography, PCR devices, enzymatic reactors, and the like. The tube (9) can also be used to supply a countercurrent flow of the leading electrolyte in an arrangement without a gel containing leading electrolyte.

In general, the gel for the leading electrolyte stabilization is formed by any uncharged material such as, for example agarose, polyacrylamide, pullulans, and the like. In some devices, the top surface is left open, or in some devices the top surface is closed, depending on the nature of the separation to be performed. If closed, the material used to cover the device is preferably a heat conducting, insulating material so as to prevent evaporation during the operation of an epitachophoresis device.

In general, the ring (circular) electrode is preferentially a gold-plated or platinum-plated stainless steel ring as this allows for maximum chemical resistance and electric field uniformity. Alternatively stainless steel and graphite electrodes may be used in some devices, particularly for disposable devices. Also, the ring (circular) electrode can be substituted with other moieties that provide similar function, e.g., by an array of wire electrodes. Moreover, a 2 dimensional array of regularly spaced electrodes may additionally or alternatively be used in epitachophoresis devices. An array of regularly spaced electrodes in a circular orientation may also be used in epitachophoresis devices. Furthermore, other electrode configurations may also be used to effect different electric field shapes based on the desired sample separation (e.g., for directing the focused zones). Such configurations are described as polygon arrangements of electrodes. When divided into electrically separated segments, a switched electric field is created for time dependent shape of the driving electric field. Such an arrangement facilitates sample collection in some devices.

### Example 2: Epitachophoresis Device Operation

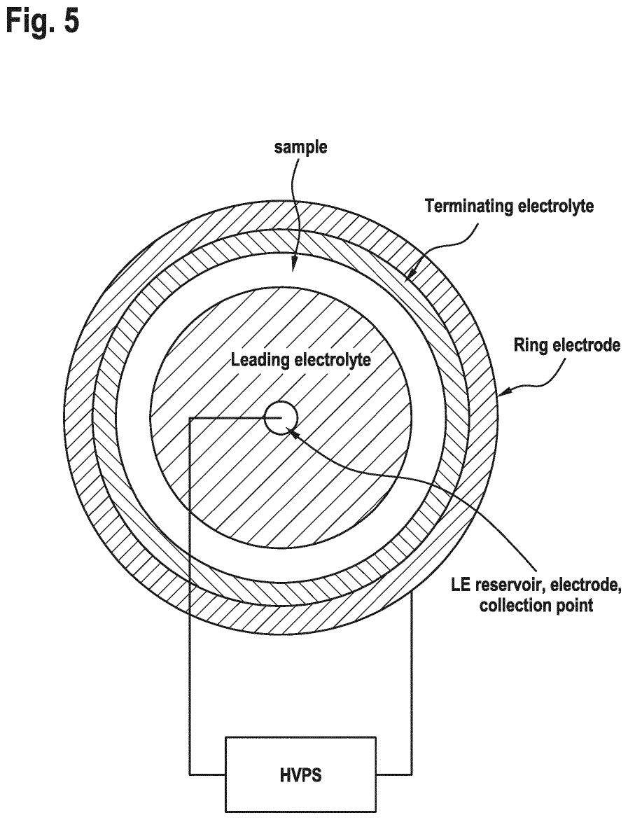

Epitachophoresis devices, such as those of the designs presented in FIG. 1-FIG. 4, are operated in either a two electrolyte reservoir arrangement, with the leading electrolyte followed by sample mixed with terminating electrolyte or with the sample mixed with the leading electrolyte followed by the terminating electrolyte, or in a three electrolyte reservoirs arrangement, as is presented in FIG. 5. In such an arrangement, the sample may be mixed with any conducting solution. Alternatively, when the sample contains suitable terminating ions the terminating electrolyte zone can be eliminated. Referring to FIG. 2A-FIG. 2B, upon filling the terminating electrolyte reservoir (2) with a mixture of sample and suitable terminating electrolyte and turning on the electric power supply (6), the ions start moving towards the center electrode (5) and form zones at the boundary between leading and terminating electrolytes (7). The concentrations of the sample zones during the migration adjust according to general isotachophoretic principles [Foret, F., Krivankova, L., Bocek, P., Capillary Zone Electrophoresis. Electrophoresis Library, (Editor Radola, B. J.) VCH, Verlagsgessellschaft, Weinheim, 1993.]. Thus, the low concentrated sample ions are concentrated and highly concentrated ones are diluted. Once the sample zone enters the electrolyte reservoir (4) the separation process is stopped, and the focused material is collected in the center of the device. In practice, final concentrations of migrating zones have a concentration comparable to that of the leading ion. Typically, concentration factors of anywhere from 2 to 1000 or even more are achieved using epitachophoresis.

In a three electrolyte reservoir arrangement, the sample is applied in between the leading and terminating electrolytes (see, for example, FIG. 5), and such an arrangement results in slightly faster sample concentration and separation as compared to a two electrolyte reservoir arrangement.

To avoid mixing, the leading electrolyte and the trailing electrolyte are stabilized by a neutral (uncharged) viscous media, e.g., agarose gel (see, for example, FIG. 2A-FIG. 2B, 3, which represents the leading electrolyte optionally contained within a gel or hydrodynamically separated from the terminating electrolyte).

All common electrolytes known to those skilled in the art that are used for isotachophoresis can be used with the present epitachophoresis devices when the leading ions have a higher effective electrophoretic mobility than that of the sample ion(s) of interest. The opposite is true for the selected terminating ions.

The device is operated either in positive mode (separation/concentration of cationic species) or in negative mode (separation/concentration of anionic species). The most common leading electrolytes for anionic separation using epitachophoresis include, for example, chloride, sulfate, or formate, buffered to desired pH with a suitable base, e.g., histidine, TRIS, creatinine, and the like. Concentrations of the leading electrolyte for epitachophoresis for anionic separation range from 5 mM-1 M with respect to the leading ion. Terminating ions then often include MES, MOPS, HEPES, TAPS, acetate, glutamate and other anions of weak acids and low mobility anions. Concentrations of the terminating electrolyte for epitachophoresis in positive mode range from: 5 mM-10 M with respect to the terminating ion.

For cationic separation common leading ions for epitachophoresis include, for example: potassium, ammonium or sodium with acetate or formate being the most common buffering counterions. Reaction hydroxonium ion moving boundary then serves as a universal terminating electrolyte formed by any weak acid.

In both positive and negative modes, the increase of the concentration of the leading ion results in proportional increase of the sample zone at the expense of increased electric current (power) for a given applied voltage. Typical concentrations are in the 10-100 mM range; however, higher concentrations are also possible.

Furthermore, in cases where only zone electrophoretic separation is sufficient, the device can be operated with only one background electrolyte.

Current and/or voltage programming is suitable for adjusting the migration velocity of the sample. It should be noted that in this concentric arrangement, the cross section area changes during the migration and the velocity of the zone movement is not constant in time. Thus, this arrangement does not strictly follow the isotachophoretic principle where the zones migrate with constant velocities. According to the mode of operation of the electric power supply (6) three basic cases may be distinguished: 1. Separation at Constant Current; 2. Separation at Constant Voltage; and 3. Separation at Constant Power.

Variables for the equations described below are as follows: d=distance migrated (d<0; r>); E=electric field strength; H=Electrolyte (gel) height; I=electric current; J=electric current density; κ=electrolyte conductivity; r=radius; S=cross-section area; u=electrophoretic mobility; v=velocity; and X=length from the center electrode to epitachophoresis boundary.

In the common mode of operation that uses constant electric current supplied by a high voltage power supply (HVPS), the migrating zone is accelerated as it moves closer to the center due to increasing current density. With regard to separation at constant current and using a device comprising a circular architecture, e.g., a device comprising one or more circular electrodes, the relative velocity at a distance, d, depends only on the mobility (conductivity) of the leading electrolyte, as is demonstrated by the derivation of the epitachophoresis boundary velocity at v at the distance d from the start radius r as follows:

General Equations:

U=IR or E=J/κ (Ohm's Law)

E=U/X (electric field strength)

$$J = E κ ⇒ I = \frac{SU κ}{X} ; R = X / κ S$$

v=uE

S=2πXH

Epitachophoresis Boundary Velocity v at the distance d from the start with radius r:

v_{(d)}=u_{L}I/2π(r−d)hκ_{L}=Constant/(r−d)

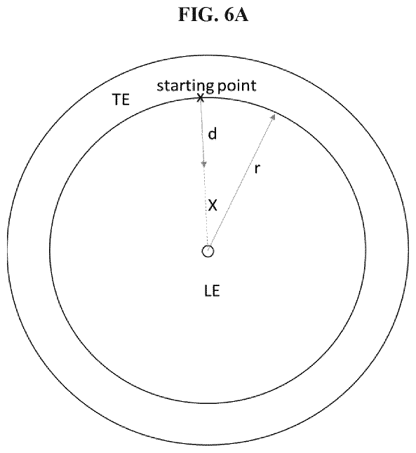

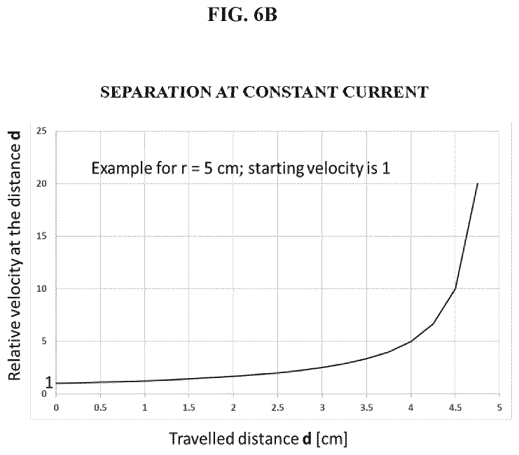

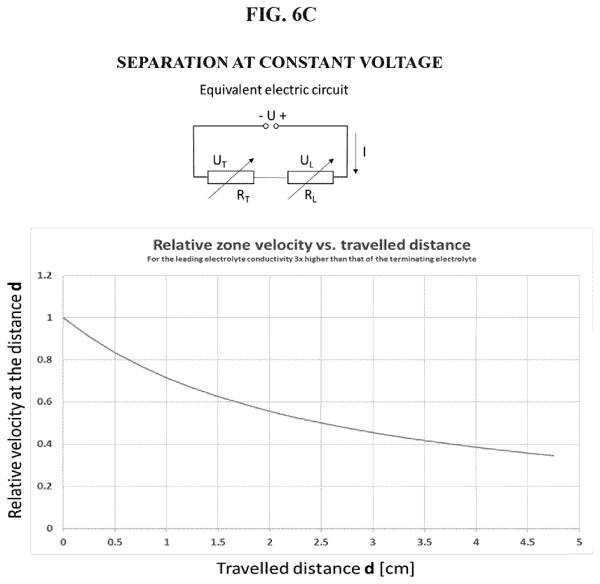

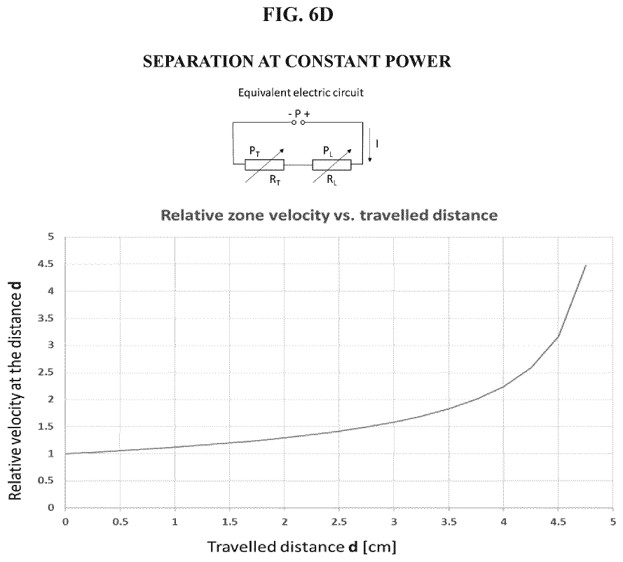

For a plot of the relationship of the distance traveled (d) vs. the relative velocity at the distance d at constant current, see FIG. 6B.

With regard to separation at constant voltage and using a device comprising a circular architecture, e.g., a device comprising one or more circular electrodes, the relative velocity at a distance, d, depends on the mobilities (conductivities) of both the LE and TE, as is demonstrated by the derivation of the epitachophoresis boundary velocity at v at the distance d from the start radius r as follows:

General Equations:

U=IR or E=J/κ (Ohm's Law)

E=U/X (electric field strength)

$$J = E κ ⇒ I = \frac{SU κ}{X} ; R = X / κ S$$

Calculation of the boundary velocity:

U_{L}=U−U_{T}=U−IR_{T}

U_{L}=U−Id/Sκ_{T}

U_{L}=U−U_{L}κ_{L}d/(r−d)κ_{T}

U_{L}=U(r−d)κ_{T}/[(r−d)κ_{T}+κ_{L}d]

E_{L}=U_{L}/(r−d)

E_{L}=Uκ_{T}/[(r−d)κ_{T}+κ_{L}d]

v_{L}=u_{L}κ_{T}/[(r−d)κ_{T}+κ_{L}d

For a plot of the relationship of the distance traveled (d) vs. the relative velocity at the distance d at constant voltage, see FIG. 6C.

With regard to separation at constant power and a device comprising a circular architecture e.g., a device comprising one or more circular electrodes, the relative velocity at a distance, d, depends on the mobilities (conductivities) of both the LE and TE, as is demonstrated by the derivation of the epitachophoresis boundary velocity at v at the distance d from the start radius r as follows:

General Equations:

P=UI=I²R (electric power)

U=IR or E=J/κ (Ohm's Law)

E=U/X (electric field strength)

J=Eκ⇒I=SUκ/X; R=X/κS

Calculation of the boundary velocity:

$$P = {P}_{L} + {P}_{T} P = {I}^{2} ( {R}_{L} + {R}_{T} ) P = {I}^{2} ( \frac{r - d}{{κ}_{L} S} + \frac{d}{{κ}_{T} S} ) ) \\ I = \sqrt{P / [ \frac{r - d}{{κ}_{L} S} + \frac{d}{{κ}_{T} S} ]} \\ {U}_{L} = {IR}_{L} = I ( r - d ) / {κ}_{L} S \\ {E}_{L} = \frac{{U}_{L}}{r - d} = I / {κ}_{L} S \\ {E}_{L} = \sqrt{P / [ ( r - d ) {κ}_{L} S + \frac{d {κ}_{L}^{2} S}{{κ}_{T}} ]}$$

κ is a small number, thus:

E_{L}≈\sqrt{P/(r−d) κ_{L}S}

For a plot of the relationship of the distance traveled (d) vs. the relative velocity at the distance d at constant power, see FIG. 6D.

### Example 3: Epitachophoresis using an Exemplary Device

An epitachophoresis device, as presented in FIG. 7, was used to perform an epitachophoresis separation that focused sulfanilic acid dye (SPADNS) into a concentric ring. 1 W constant power was applied to effect epitachophoresis in the epitachophoresis device.

Referring to FIG. 7, SPADNS was focused into a concentric ring-shaped focused zone, which can be seen as the red zone of FIG. 7. The upper half of the red circle showed that the height of the zone was approximately 5 mm. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

### Example 4: Epitachophoresis using an Exemplary Device

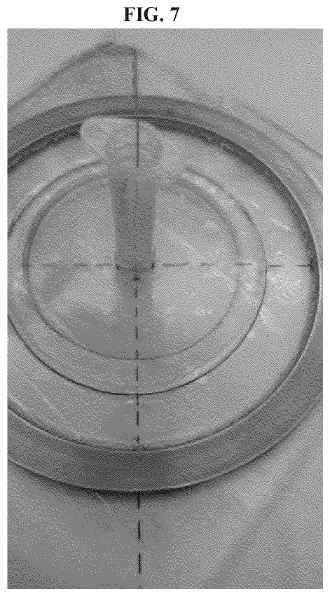

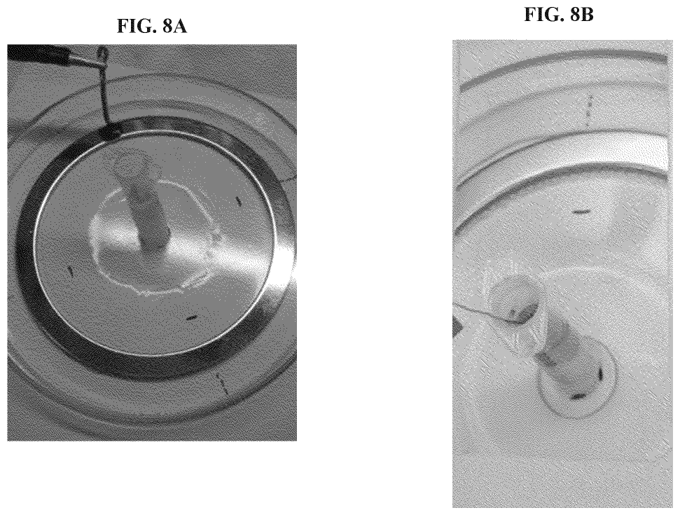

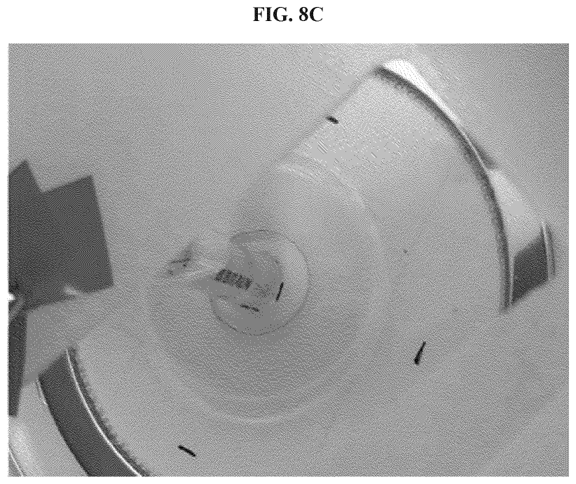

An epitachophoresis device (FIG. 8A) was used to perform epitachophoresis to focus sulfanilic acid (SPADNS). The device of FIG. 8A had a circular architecture and a circular gold electrode with a diameter of 10.2 cm. HCl-histidine (pH 6.25) was used as the leading electrolyte and was contained in 10 mL of an 0.3% agarose gel which had a diameter of 5.8 cm. 15 mL of MES Tris (pH 8.00) was used as the trailing electrolyte. The syringe reservoir of the device contained the leading electrolyte HCl His (pH 6.25). 300 μl of SPADNS at a concentration of 0.137 mM was prepared in trailing electrolyte and loaded into the device. To effect epitachophoresis, a constant power of 1 W was used.

Referring to FIG. 8B, SPADNS was focused into a concentric ring-shaped focused zone, which can be seen as the red zone of FIG. 8B. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

Furthermore, the epitachophoresis device of FIG. 8A was used to perform epitachophoresis to focus a 30 nt oligomer (ROX-oligo). The device of FIG. 8A had a circular architecture and a circular gold electrode with a diameter of 10.2 cm. 10 mM HCl-histidine (pH 6.25) was used as the leading electrolyte was contained in 10 mL of an 0.3% agarose gel which had a diameter of 5.8 cm. 15 mL of 10 mM MES Tris (pH 8.00) was used as the trailing electrolyte. The syringe reservoir of the device contained the leading electrolyte HCl His (pH 6.25) at a concentration of 100 mM. 75 μl of ROX-oligo at a concentration of 100 μM was prepared in trailing electrolyte and loaded into the device. To effect epitachophoresis, a constant power of 1 W was used.

Referring to FIG. 8C, ROX-oligo was focused into a concentric ring-shaped focused zone, which can be seen as the blue zone of FIG. 8C. As the epitachophoresis zone moved from the edge towards the center of the device, eventually the focused zone of the ROX-oligo entered the center of the device and was collected in the center of the device, thereby demonstrating focusing and recovery of a desired sample using epitachophoresis.

### Example 5: Epitachophoresis using an Exemplary Device

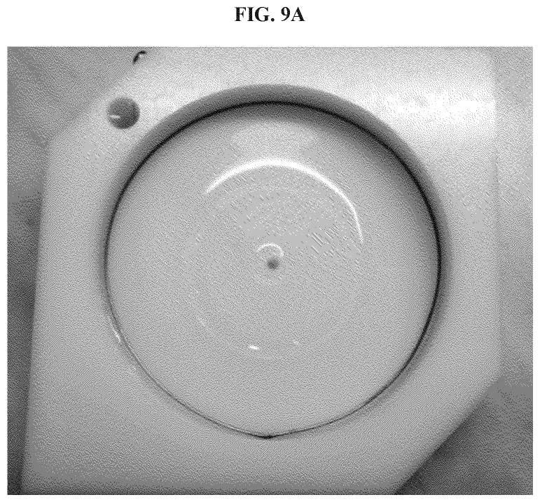

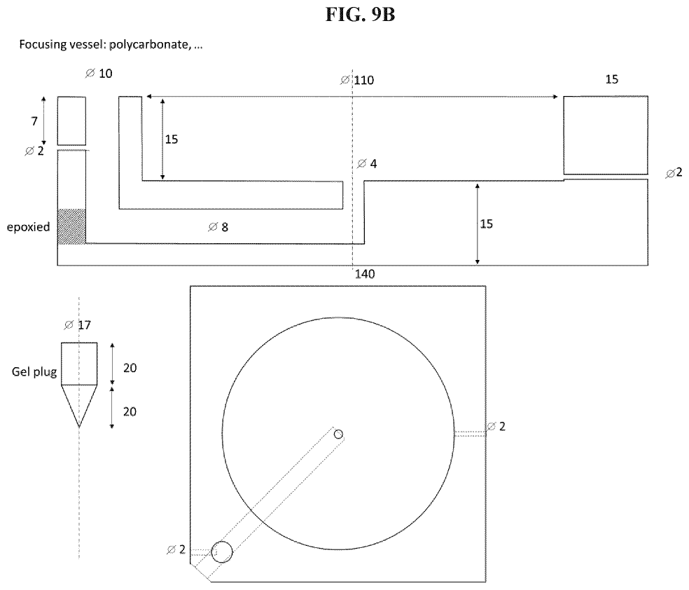

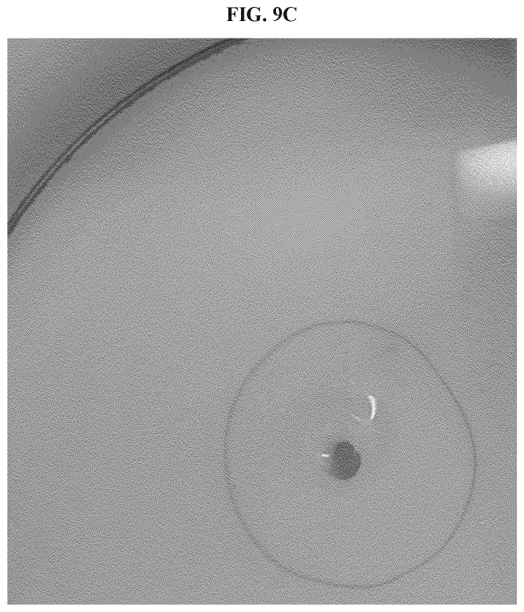

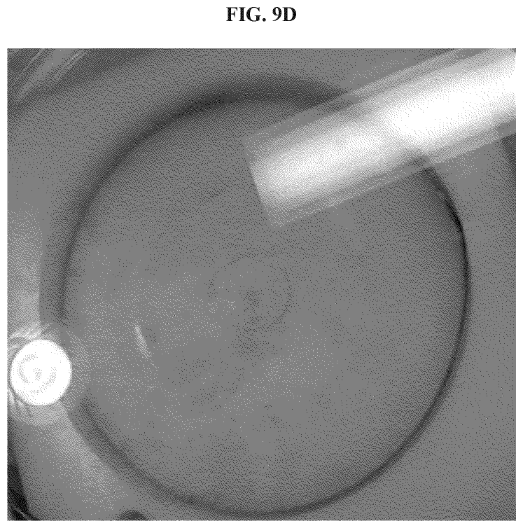

An epitachophoresis device (FIG. 9A-FIG. 9B) was used to perform epitachophoresis to focus sulfanilic acid dye (SPADNS), which was subsequently collected from said device (FIG. 9C-FIG. 9D). The device of FIG. 9A-FIG. 9B had a circular architecture and a circular stainless steel wire electrode with a diameter of 11.0 cm. Referring to FIG. 9B, the numbers of the schematic represent dimensions in millimeters. 20 mM HCl-histidine (pH 6.20) was used as the leading electrolyte. Either 5 mL of 10 mM MES Tris (pH 8.00) was used as trailing electrolyte with an 0.3% agarose gel in LE, wherein the gel had a diameter of 8.9 cm (FIG. 9C) and was formed prior to introduction of TE, or 15 mL of 10 mM MES Tris (pH 8.00) was used as trailing electrolyte contained in an 0.3% gel which had a diameter of 5.8 cm (FIG. 9D) and was formed prior to introduction of TE. The electrode reservoir of the device contained leading electrolyte HCl His (pH 6.25) at a concentration of 100 mM.

Referring to FIG. 9C, 150 μl of SPADNS at a concentration of 0.137 mM was prepared in 15 mL of trailing electrolyte and loaded into the device. To effect epitachophoresis, a constant power of 2 W was used. SPADNS was focused into a concentric ring-shaped focused zone, which can be seen as the red zone of FIG. 9C. As the epitachophoresis zone moved from the edge towards the center of the device, eventually the focused zone of the SPADNS entered the center of the device and was collected in the center of the device, thereby demonstrating focusing and recovery of a desired sample using epitachophoresis. The recovered SPADNS had a 40-fold absorbance increase as compared to the absorbance of the initial 15 mL SPADNS-containing sample.

Referring to FIG. 9D, 150 μl of SPADNS at a concentration of 0.137 mM was prepared in 15 mL of trailing electrolyte and loaded into the device. To effect epitachophoresis, a constant power of 2 W was used. SPADNS was focused into a concentric ring-shaped focused zone, which can be seen as the red zone of FIG. 9D. As the epitachophoresis zone moved from the edge towards the center of the device, eventually the focused zone of the SPADNS entered the center of the device and was collected in the center of the device, thereby demonstrating focusing and recovery of a desired sample using epitachophoresis. The recovered SPADNS had a 40-fold absorbance increase as compared to the absorbance of the initial 15 mL SPADNS-containing sample.

The epitachophoresis device of FIG. 9A-FIG. 9B was also used to perform epitachophoresis to focus SPADNS from a physiological saline solution in a device that did not use a gel. 20 mM HCl-histidine (pH 6.20) was used as the leading electrolyte. 13 mL of 10 mM MES Tris (pH 8.00) was used as trailing electrolyte, which was further mixed with 3 mL of 0.9% NaCl. The electrode reservoir of the device contained leading electrolyte HCl Histidine (pH 6.25) at a concentration of 100 mM.

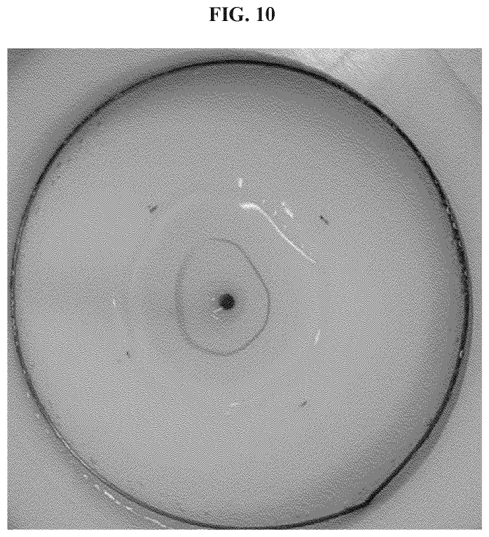

Referring to FIG. 10, 150 μl of SPADNS at a concentration of 0.137 mM was prepared in 13 mL of trailing electrolyte mixed with 3 mL of 0.9% NaCl and loaded into the device. To effect epitachophoresis, a constant power of 2 W was used. SPADNS was focused into a concentric ring-shaped focused zone, which can be seen as the red zone of FIG. 10. As the epitachophoresis zone moved from the edge towards the center of the device, eventually the focused zone of the SPADNS entered the center of the device and was collected in the center of the device, thereby demonstrating focusing and recovery of a desired sample using epitachophoresis.

The epitachophoresis device of FIG. 9A-FIG. 9B was also used to perform epitachophoresis to separate and to focus SPADNS and Patent Blue dye with acetic acid as a spacer. 20 mM HCl-histidine (pH 6.20) was used as the leading electrolyte. 5 mL of 10 mM MES Tris (pH 8.00) was used as trailing electrolyte, which was further mixed with 150 μl of 10 mm acetic acid, 150 μl of 0.1 mM Patent Blue dye, and 150 μl of 0.137 mM SPADNS. The effective mobility values (10⁻⁹ m²/Vs) of SPADNS, acetic acid, and Patent Blue dye were 55, 42,7, and 32, respectively. The electrode reservoir of the device contained leading electrolyte HCl His (pH 6.25) at a concentration of 100 mM. No gel was used in the channel of this device for this experiment, however gel was present on the top of the device platform.

Referring to FIG. 11, the mixture of trailing electrolyte, SPADNs, acetic acid, and Patent Blue dye was loaded into the device. To effect epitachophoresis, a constant power of 2 W was used. SPADNS was focused into a concentric ring-shaped focused zone, which can be seen as the red zone/inner zone of FIG. 11, and Patent Blue dye was focused into a concentric ring-shaped focused zone as well, which can be seen as the blue zone/outer zone of FIG. 11. As the epitachophoresis zones moved from the edge towards the center of the device, eventually the focused zones of the SPADNS and the Patent Blue dye entered the center of the device sequentially and may be collected separately in the center of the device, thereby demonstrating separation, focusing and recovery of a desired samples using epitachophoresis.

### Example 6: Device for Epitachophoresis

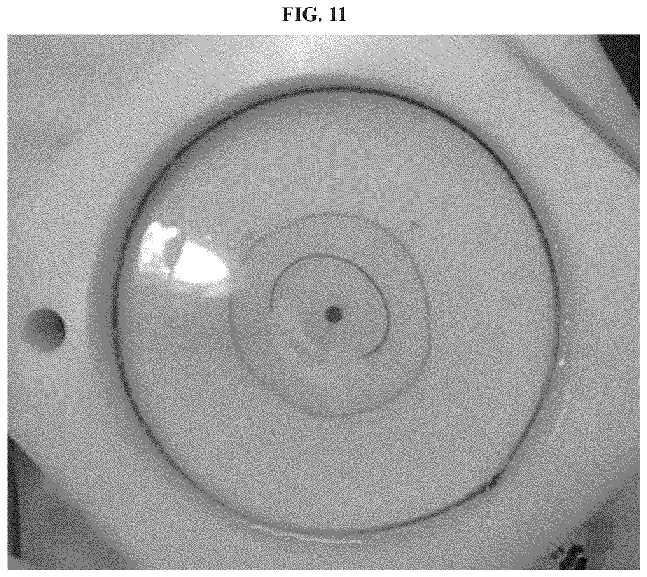

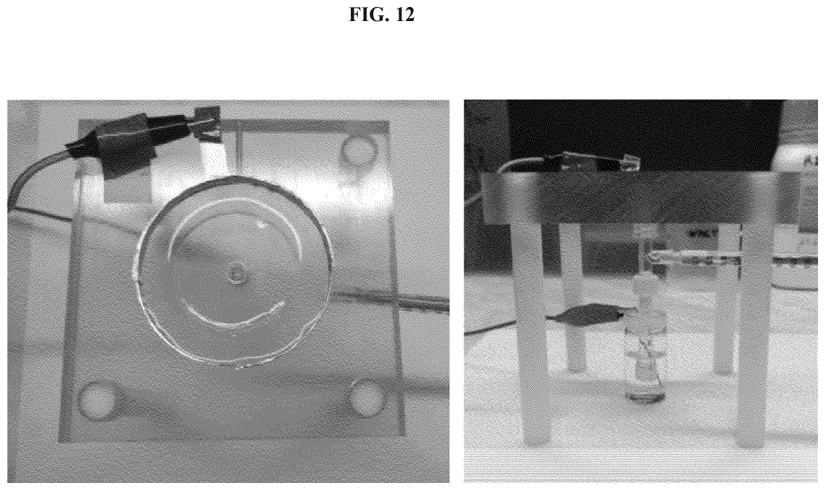

An epitachophoresis device was designed for effecting epitachophoresis (FIG. 12). The device of FIG. 12 had a circular architecture and a circular copper tape electrode with a diameter of 5.8 cm.

### Example 7: System for Effecting Epitachophoresis with Conductivity-Based Sample Detection

### Device Construction

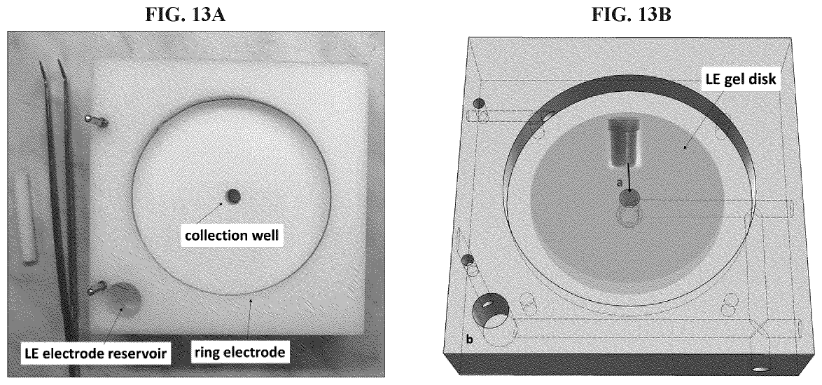

In accordance with previous examples, an epitachophoresis device with a large sample volume capacity was machined with a circular separation channel. FIG. 13A and FIG. 13B show two views of the structure of the device. The wire ring electrode (1 mm diameter stainless steel wire; radius of 55 mm) was attached on the edge of the circular separation compartment. The sample volume was defined by the space between the ring electrode and an agarose stabilized leading electrolyte disk (radius of 35 mm). Thus, the applicable sample volume was 5.7 ml for every mm of its height. The second electrode was placed in the leading electrolyte reservoir on the side of the device. The ring electrode was connected to the upper banana type connector shown in FIG. 13A. The bottom banana connector was attached to a 3 cm long, 0.4 mm diameter platinum wire electrode positioned in the leading electrode reservoir (“b” in the scheme shown in FIG. 13B). To prevent possible interferences from electrolysis products, migrating out from the leading electrolyte reservoir towards the center collection well (“a” in the scheme shown in FIG. 13B), a 9 mm ID internal channel with a total length of 20 cm long was drilled inside the device. The side openings after drilling of the device were plugged by silicon septa. The central collection well with the diameter of 9 mm was drilled through the device and closed from the bottom by a moving Ertacetal® rod sealed by a rubber O-ring.

For every analysis, a plastic vial with a semipermeable membrane (Slide-A-Lyzer™ MINI Dialysis Units 2000 Da MWCO, Thermo Fisher Scientific, USA) was inserted into the central collection well after filling it with the leading electrolyte all the way to the leading electrode reservoir. To minimize the volume, the Slide-A-Lyser was cut in half by a razor blade creating a collection cup with a volume of less than 200 microliters. Next a 0.3% agarose gel disk (70 mm diameter, 4 mm thick) with a central 8 mm hole was prepared in the leading electrolyte, positioned in the center of the device and covered by a 75×1 mm round glass plate also having a center 8 mm hole to avoid bubble accumulation. Although various electrophoretic separation modes may be applied (e.g., zone electrophoresis, isoelectric focusing, or displacement electrophoresis), we have used epitachophoresis with an electrolyte system comprising leading (LE) and terminating (TE) electrolytes. The sample solution in the terminating electrolyte was applied by a syringe into the space between the gel disk and ring electrode. The polarity of the electric current connection was selected so that anionic sample components migrated from the ring electrode towards the collection well in the device center. After the focused sample zone entered the collection cup, the electric current was turned off, and the sample was pipetted out for further use. The empty collection cup was lifted up by the moving rod and discarded.

### Electrodriven Separation Conditions

Separations were performed in negative mode, where Cl⁻ ion served as leading ion (effective mobility 79.1×10⁻⁹ m² V⁻¹ s⁻¹). Leading electrolyte (LE) contained 100 mM HCl-Histidine buffer at pH 6.2 and terminating electrolyte (TE) contained 10 mM TAPS titrated by TRIS to pH 8.30. The agarose stabilized leading electrolyte disk was prepared in 20 mM leading electrolyte (HCl-Histidine; pH 6.25). All buffers were prepared in deionized water. The power supply was provided by a PowerPac 3000 (BioRad), which was run at constant power mode at 2 W (this corresponds approximately to 16 mA and 120 V at the beginning of the analysis). Analysis took approximately 1 hour (≈10 mA and 200 V at the end of analysis).

### Sample Detection

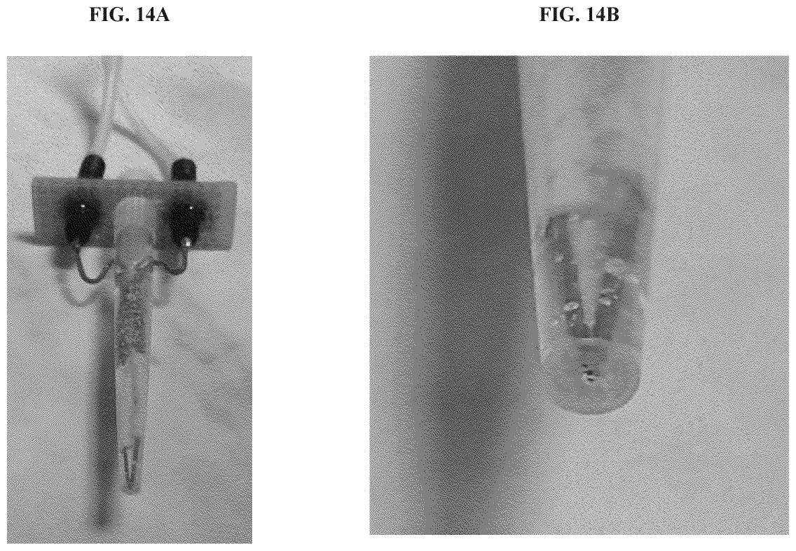

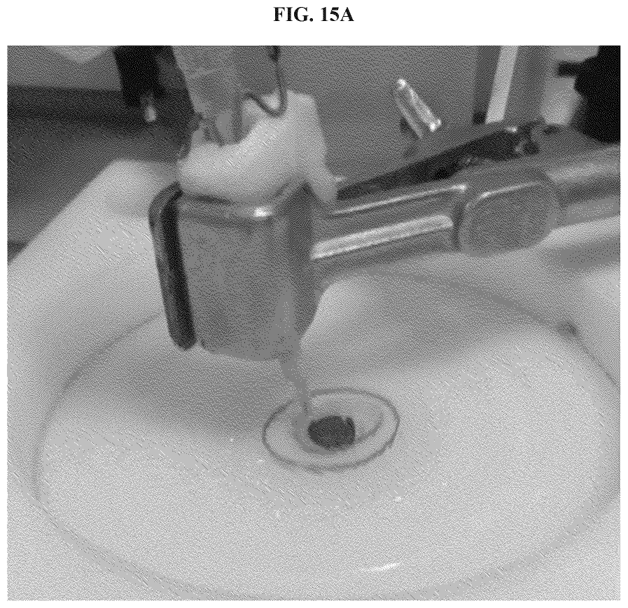

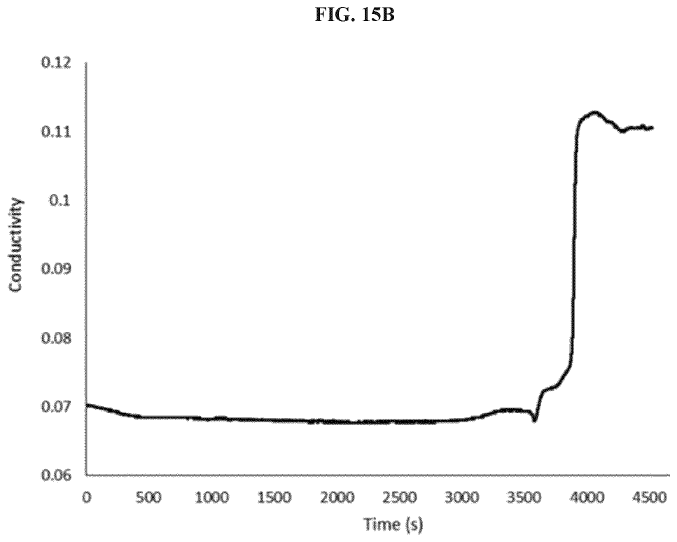

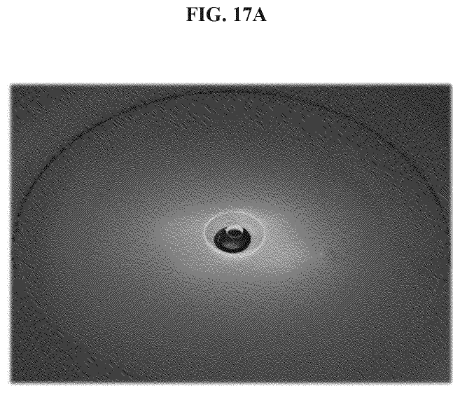

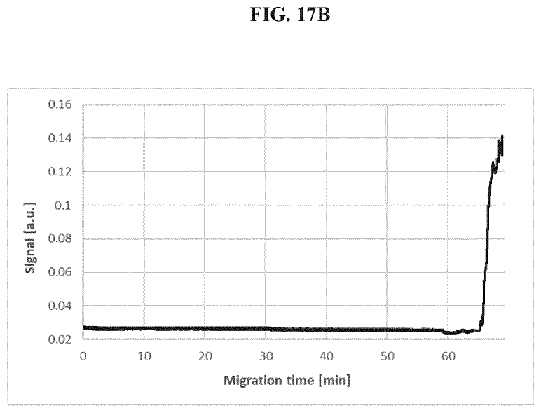

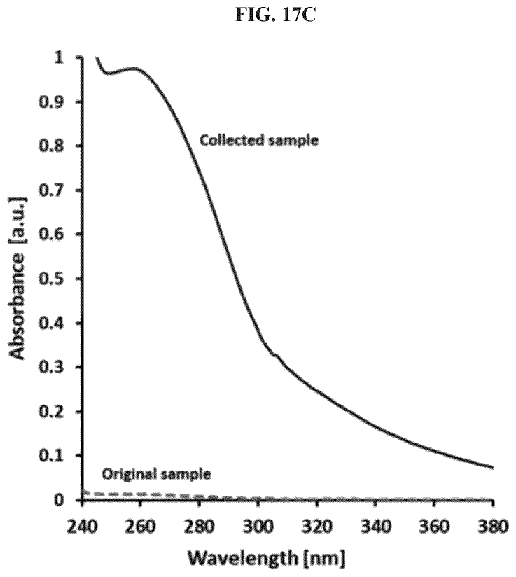

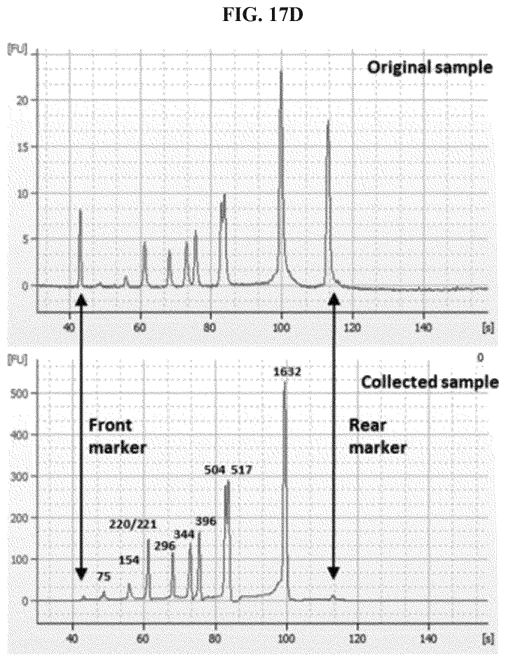

For detecting the samples, a surface resistivity detection cell was constructed and connected to the conductivity detector of a commercial ITP instrument (Villa Labeco, Sp.N.Ves, Slovakia). The detection cell was prepared as follows: two platinum (Pt) wires (300 μm×2 cm long) were attached to connectors matching the ITP instrument. The opposite ends of the Pt wires were inserted into a 1 mL pipette tip, which was then filled by a quick setting epoxy resin. Finally, 1 mm of the pipette tip with the epoxy wires embedded inside was cut by a razor blade exposing flat epoxy surface with two round Pt electrodes. See FIG. 14A and FIG. 14B. The detection cell was mounted in a laboratory stand gently touching the surface of the agarose gel disk close to the collection vial, as exemplified in FIG. 15A. This system for detection was employed to generate the conductivity trace of FIG. 15B and FIG. 17B.

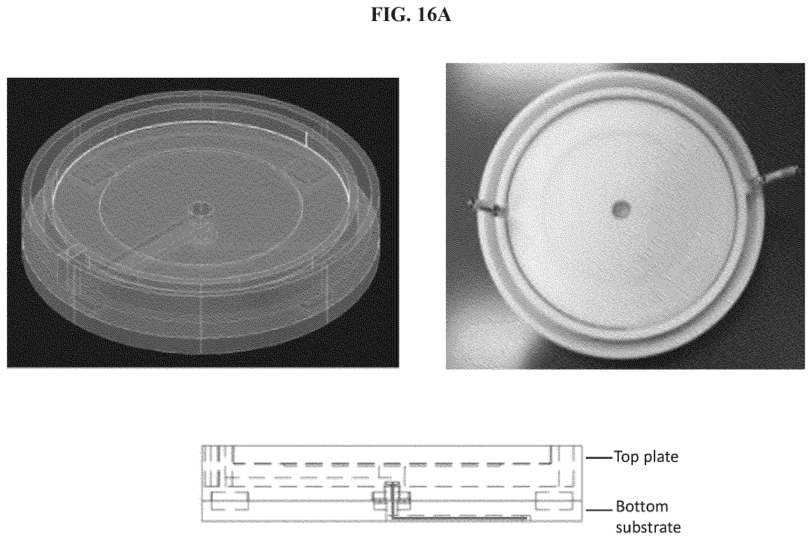

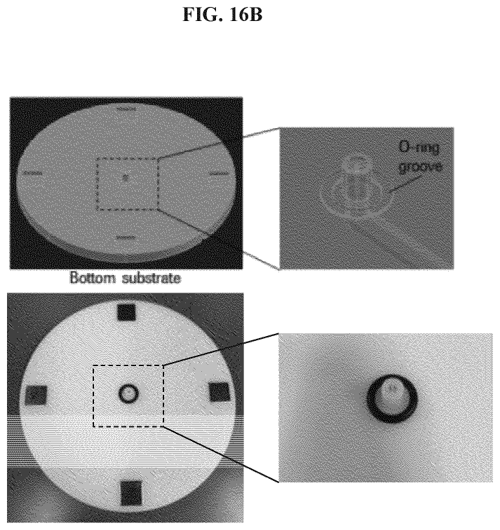

Another exemplary system was also constructed for sample detection during epitachophoresis. In this system, surface resistivity detection probes consisting of two platinum (Pt) wires with a diameter of 500 μm were incorporated within the bottom substrate (i.e., bottom plate) of an epitachophoresis device, as shown in FIG. 16A and FIG. 16B. The tips of the wires were within proximity of the semipermeable membrane from the bottom via dedicated channels within the central pillar on the bottom substrate. The opposite ends of the wires were connected to the conductivity detector of a commercial ITP instrument (Villa Labeco, Sp.N.Ves, Slovakia). The top plate, serving as the epitachophoresis device, was assembled with the bottom substrate using magnets, while the o-ring (see FIG. 16B) enabled complete sealing between the two substrates in order to prevent any leakage.

### Chemicals

Buffer components: L-histidine monohydrochloride monohydrate (99%), L-histidine (99%), N-tri s(hydroxymethyl)methyl-3-aminopropanesulfonic acid (TAPS; 99.5%) and tris-(hydroxymethyl)aminomethane (TRIS; 99.8%) were purchased from Sigma-Aldrich (USA). Agarose NEEO ultra-quality Roti®garose with low electroendosmosis was purchased from Carl Roth (Germany). Acetic acid and anionic dye Patent blue V sodium salt were from Sigma-Aldrich; red anionic dye SPADNS (1,8-dihydroxy-2-(4-sulfophenylazo)naphthalene-3,6-disulfonic acid trisodium salt was from Lachema, Brno, Czech Republic.

### Focusing of SPADNS and Patent Blue

To test the above-described exemplary device comprising epitachophoresis and electrical sample detection, the device was used to focus and detect test analytes: SPADNS and Patent Blue. Leading electrolyte (LE): HCl-HIS buffered to pH 6.2. Trailing electrolyte (TE): TAPS-TRIS buffered to pH 8.3. The gel was formed from 20 ml of polyacrylamide gel 6% in 20 mM LE. 100 mM LE was added to the electrode reservoir. Sample solution: 15 ml of 10 mM TE+150 μL 0.1 mM SPADNS+150 μL 0.1 mM Patent blue. The sample solution in the terminating electrolyte was applied by a syringe into the space between the gel disk and ring electrode. The device was run in constant power mode with P=2 W. FIG. 15A provides an image of the focusing of the SPADNS and Patent Blue, showing the conductivity sample detection near the sample collection well. FIG. 15B provides the conductivity trace for this sample focusing and shows a marked change in conductivity/resistivity that was due to the transition between LE and TE, which encompassed the focused zone of sample (SPADNS and Patent blue).

### DNA Analysis

Low molecular weight dsDNA ladder labeled with Fluorescein (ten fragments from 75 base pairs—bp to 1622 bp) was from Bio-Rad, USA. The DNA concentration in the collected fraction was evaluated using Qubit fluorometer (Invitrogen, Carlsbad, CA, USA) by using the high sensitivity dsDNA Qubit quantitation assay kit. The concentration of the target molecule in the sample was reported by a fluorescent dye emitting only when bound to the DNA. The collected fractions were further analyzed using the chip CGE-LIF instrument Agilent 2100 Bioanalyzer (Agilent, Santa Clara, CA, United States). This analysis provided size information of the collected DNA fragments in the sample using the high sensitivity DNA reagent kit (Agilent, United States).

### DNA Focusing

Electrophoretic mobilities of DNA fragments above 50 bp are approximately 37×10⁻⁹ m²/Vs in free solution, and short fragments (˜20-50 bp) may deviate by only ˜10%. Based on these mobilities, we designed a discontinuous electrolyte system suitable for focusing of all sample DNA fragments into a single concentrated zone.

For experimental testing, we selected a fluorescein labeled low molecular range DNA ladder with fragment sizes ranging from 75 to 1632 bp. The fluorescence of the sample with only one fluorophore per DNA fragment was illuminated by a 2 cm radius laser beam (FIG. 17A). The surface resistivity detection was used to indicate the transition of the LE/TE boundary close to the collection well (conductivity trace shown in FIG. 17B). The overall change in resistivity from LE to TE was used as an indicator of the sample location. Based on this change, the voltage was turned off and separation was stopped. The collected fraction was analyzed by both UV spectrometry (absorbance measurements) (FIG. 17C) and Bioanalyzer-based analysis (FIG. 17D). The lower signal intensity of the front and rear markers in FIG. 17D (added at the same amount to the initial sample and to the final sample according the manufacturer's instructions) was due to the higher DNA concentration in the collected fraction. In both cases the ˜30× concentration increase in the collected fraction corresponded to the decrease of the sample volume from the starting 15 mL to the sample collection volume of 280 μL in this exemplary embodiment. The volume of the migrating DNA zone prior to entering the collection cup was much smaller (−3 μL) and the final fraction concentration depends mainly on the volume of the selected collection vial.

### Example 8: ETP with Voltage-based Sample Detection

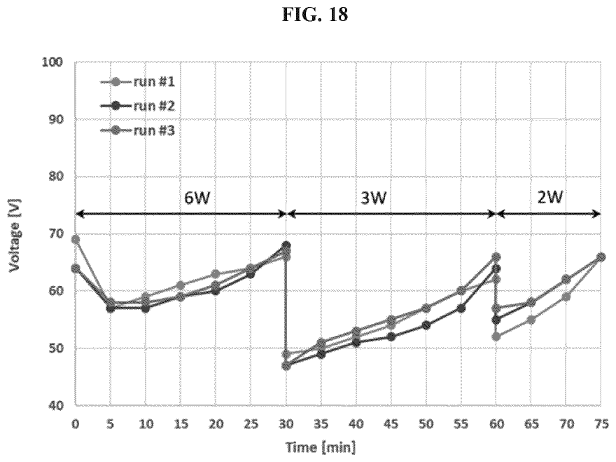

In the present example, three independent ETP runs were performed to focus and collect cfDNA from 1 mL of plasma using an ETP device, and the voltage was measured during the time course of each of the three independent ETP runs. Each of the three ETP runs was 75 minutes in length. The power level at the beginning of each ETP run was 6 W. The power level was subsequently lowered to 3 W at 30 min., and then lowered to 2 W at 60 min. The results obtained during each of the three independent ETP runs are presented in FIG. 18.

Referring now to FIG. 18, in each stage during which the power was kept constant, the voltage gradually increased. In the last stage (2 W power, 60 min. to 75 min.), the voltage was 65 V when the focused zone comprising the nucleic acid molecules migrated into the collection cup. Voltage profiles were observed to be consistent when comparing each of the three independent runs, which indicated that the voltage feedback from the power supply could be used to monitor the location of the nucleic acid molecules within the device.

### Example 9: ETP with Optical-based Sample Detection

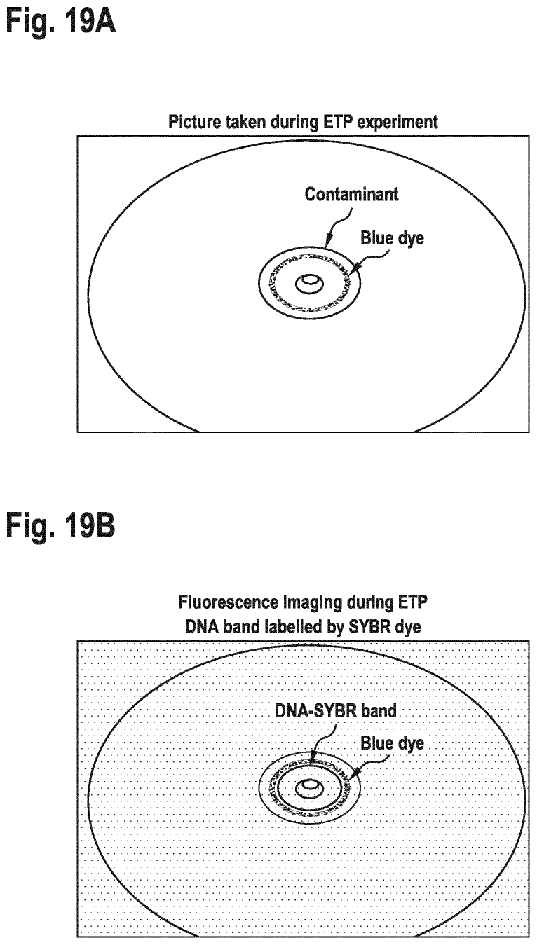

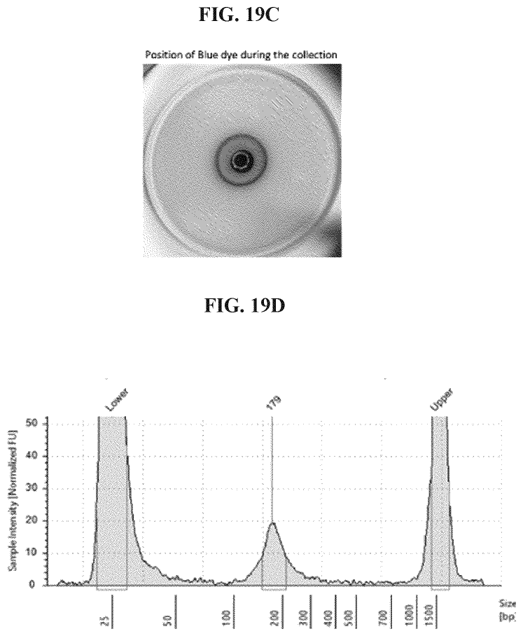

In the present example, ETP runs were performed using optical detection with colored dye to monitor the position of nucleic acids during the ETP runs. A chromatic dye with an electrophoretic mobility lower than nucleic acids can be used to enable optical tracking of the location of nucleic acids. For example, such dyes include Brilliant blue FCF, Indigo carmine, Sunset yellow FCF, Allura red, Fast green FCF, Patent blue V and Carmoisine. In the present example, two independent ETP runs were performed to focus and collect nucleic acid molecules using brilliant blue dye as an optical marker (see ETP Run 1: FIG. 19A-FIG. 19B; and ETP Run 2: FIG. 19C-FIG. 19D).

FIG. 19A presents an image of an ETP device during an ETP run in which Brilliant Blue dye was used as an optical marker during focusing and collection of nucleic acid molecules, and also in which SYBR-gold dye was further used to monitor the position of the nucleic acid molecules. The electrophoretic mobility of the blue dye was lower than that of the nucleic acids, and, as such, the blue dye migrated after the focused zone comprising the nucleic acid. Contaminant appeared as a brown-colored focused zone (see FIG. 19A). In addition to the photographic image of FIG. 19A, a fluorescence-based image was taken during the ETP run (see FIG. 19B). The fluorescence-based image of FIG. 19B demonstrates that the focused zone comprising the band comprising DNA labeled with SYBR-gold migrated faster than the brilliant blue dye.

FIG. 19C presents an image of an ETP device during an ETP run in which Brilliant Blue was used as an optical marker during focusing and collection of nucleic acid molecules. A plasma sample comprising cfDNA was used for the ETP run of FIG. 19C and FIG. 19D. The ETP run was stopped once the dye band reached the collection well. After focusing and collecting the cfDNA, analysis was performed on the focused and collected cfDNA sample using an Agilent TapeStation system (see FIG. 19D). The electropherogram (see FIG. 19D) showed a peak at 179 bp representing the desired cfDNA molecules which were focused and collected during the ETP run, thereby demonstrating the utility of Brilliant blue dye to monitor the location of nucleic acids.

### Example 10: ETP with Thermal-based Sample Detection

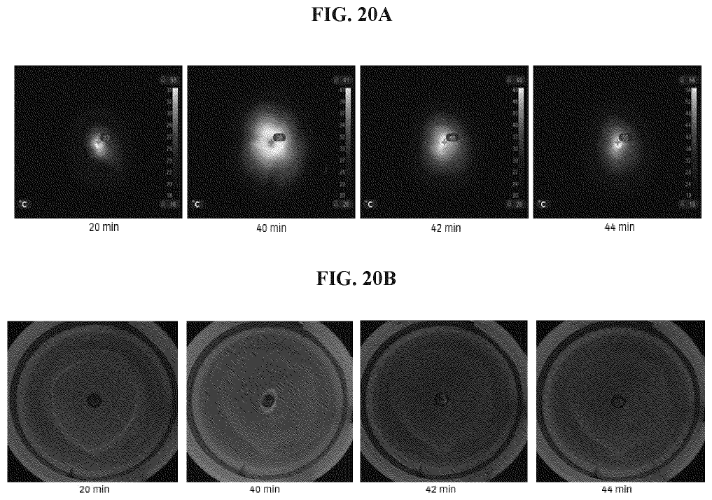

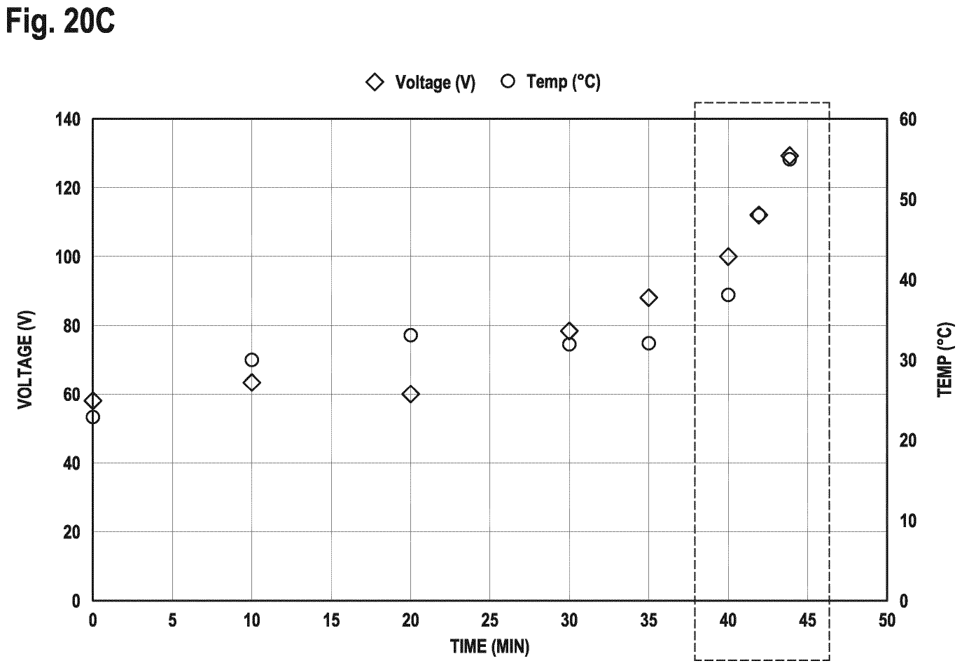

In the present example, an ETP run was performed in which thermal imaging was used during focusing and collection of a DNA ladder. For the thermal imaging of the present example, an infrared-based thermal imaging camera (SEEK thermal ShotPro) was used. Additionally, fluorescence imaging was used. Thermal and fluorescent images were taken at four sequential time points of 20 min., 40 min., 42 min., and 44 min. (see FIG. 20A-FIG. 20B). Furthermore, the voltage feedback from the power supply was measured during the ETP run.

FIG. 20A presents thermal images that were taken at four time points during the ETP run: 20 min., 40 min., 42 min., and 44 min. It was observed that the temperature at the center of the device increased by 17° C. (from 38° C. to 55° C.) between 40 and 44 min., during which the DNA ladder, visible as the green-fluorescent ring in FIG. 20B, moved into the center collection cup.

FIG. 20C presents the voltage and temperature change over time during the ETP run of the present example. It was observed that the trend of the voltage change over time was similar to that of the temperature change over time. As such, the voltage feedback from the power supply provided an additional means for monitoring the location of DNA ladder molecules within the device.

### Example 11: Total Nucleic Acid Isolation/Purification by ETP

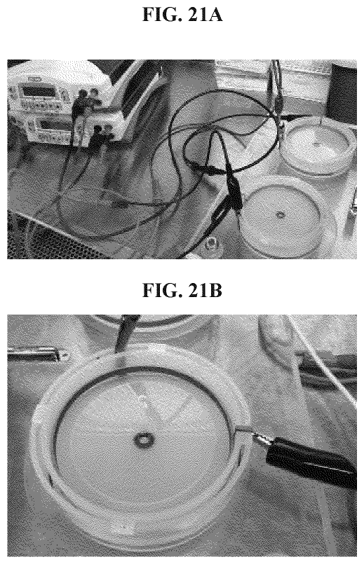

An epitachophoresis device and experimental setup (see FIG. 21A-FIG. 21B) were used to perform ETP to effect isolation (purification) of nucleic acids comprising DNA and RNA from samples comprising Formalin-Fixed Paraffin-Embedded Tissue (“FFPET”) samples.

Prior to setting up and performing ETP to isolate/purify the nucleic acids from the FFPET samples, ETP buffers, the agarose gel of the ETP device, and the shortened dialysis unit were prepared. The leading electrolyte (“LE”) buffer, which comprised HCl-Histidine pH 6.25, was prepared, and the trailing electrolyte (“TE”) buffer, which comprised TAPS-Tris pH 8.30, was prepared. The agarose gel used with the ETP device was prepared by mixing an amount of agarose appropriate for a desired agarose percentage gel with LE buffer in an Erlenmeyer flask.

Also, prior to setting up and performing ETP to isolate/purify the nucleic acids from the FFPET samples, the FFPET samples were prepared by deparaffinization and lysing of FFPET curls. As discussed further in the examples infra, in some instances the deparaffinization and lysing of the FFPET curls was performed using a Promega-based protocol, and in some instances the deparaffinization and lysing of the FFPET curls was performed using a KAPA Express Extract-based protocol.

ETP-based isolation/purification of the nucleic acids from FFPET samples generally proceeded as follows. First, the ETP device was prepared for sample loading.

An FFPET sample solution, generally containing 7 mL of TE buffer and approximately 110 to 220 ul of the lysed FFPET samples, prepared as described above, was prepared and pipetted into the gap between the gel and the circular electrode (see FIG. 21A-FIG. 21B). In instances in which Brilliant Blue dye was used as a marker, 30 ul of Brilliant Blue solution was added.

The power supply was then prepared by plugging the ETP device into the power supply. The power supply was set to a constant power of 2 W, and ETP was effected for approximately 30-40 min. by turning on the power supply.

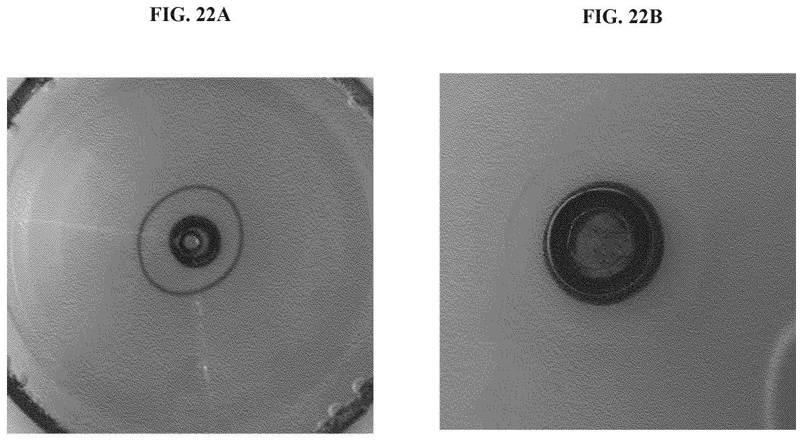

In some instances, the sample was monitored and collected as follows. In instances in which Brilliant Blue dye was used, movement of the dye was monitored using white light (no filter) (see FIG. 22A-FIG. 22B). Once the dye had completely entered the dialysis cup, the ETP run was stopped. The TE buffer and gel were removed, and the ETP isolated/purified sample was collected from the dialysis unit.

In some instances, a clean-up step was performed on the isolated/purified sample. In these instances, the entire isolated/purified ETP sample was added to an Amicon Centrifugal filter, such as a 3 kDa or 10 kDa cut off filter Amicon Centrifugal filter, and centrifugation-based cleanup was performed. Following the centrifugation-based clean up, the concentrated nucleic acid sample was collected, which sample was optionally stored at −80° C. prior to future use.

In some instances, analysis of the collected nucleic acids, e.g., DNA and/or RNA, was performed by using a Qubit fluorimeter (Invitrogen, Carlsbad, CA, USA), which reported the concentration of the nucleic acids. In some instances, analysis of the collected nucleic acids, e.g., DNA and/or RNA, was performed by using an Agilent TapeStation system (Agilent, Santa Clara, CA, United States). This analysis provided size information regarding the collected nucleic acid fragments. In some instances, analysis of the collected nucleic acids, e.g., DNA and/or RNA, was performed using a qPCR-based analysis in which Q scores, representative of nucleic acid quality, were obtained. It is noted that Q scores range from 0 (low quality) to 1 (high quality).

### Example 12: Total Nucleic Acid Isolation/Purification from FFPET Sample by ETP, dsDNA Yield Analysis, and RNA Yield Analysis

In the present example, ETP-based isolation/purification was used to isolate/purify the total nucleic acid content FFPET samples from four different CRC blocks (CRC Block #1-CRC Block #4) and from two samples of healthy adjacent tissue, and the yield of dsDNA and the yield of RNA from said ETP-based isolation and purification was compared to the dsDNA and the RNA obtained using exclusively a Promega-column based method as generally described in Example 8. dsDNA yield and RNA yield were quantified using a Qubit fluorimeter. 2-4 duplicates were performed of each run, and statistically significant results were those with P<0.03.

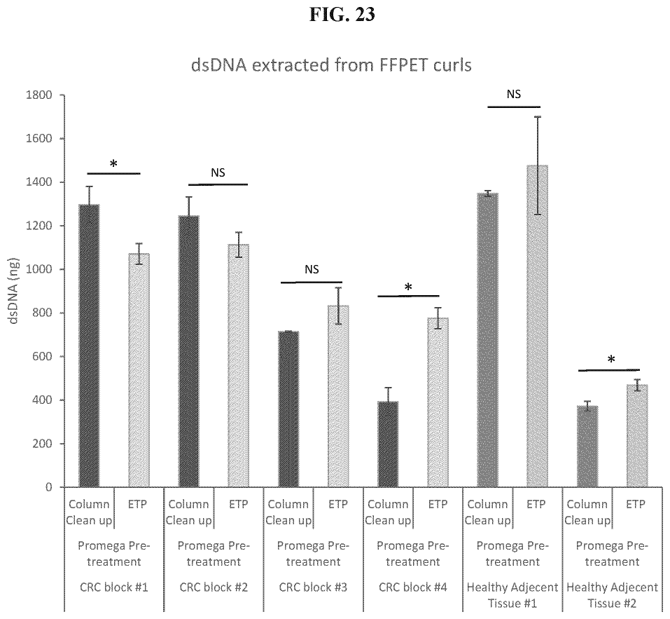

Referring now to FIG. 23, ETP-based isolation/purification of dsDNA from CRC blocks 1-4 and the healthy adjacent tissue yielded desirable levels of dsDNA in each instance. Moreover, it was observed that ETP-based isolation/purification of dsDNA outperformed the Promega-column based method significantly in instances where nucleic acid input was low, e.g., CRC block 3, CRC block 4, Healthy adjacent tissue #1, and Healthy Adjacent Tissue #2. Moreover, it is noted that the results for CRC block 4 and healthy adjacent tissue 2, as well as CRC block 1, represented statistically significant results for P<0.03.

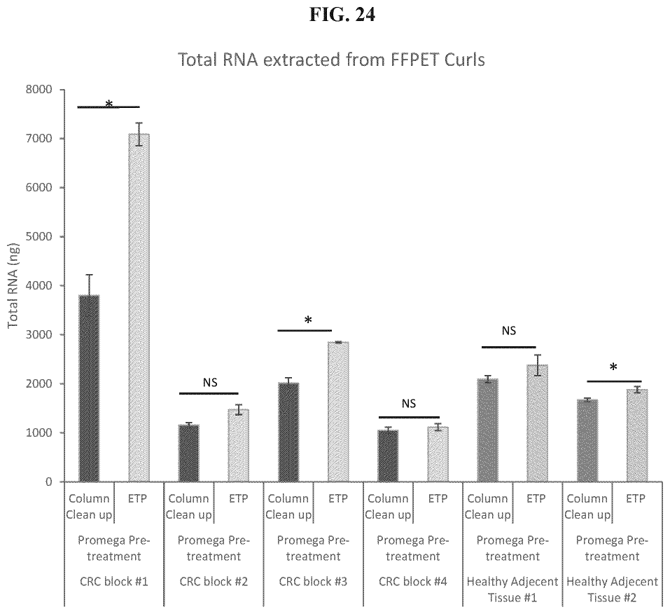

Referring now to FIG. 24, ETP-based isolation/purification of RNA from CRC blocks 1-4 and the healthy adjacent tissue samples yielded desirable levels of RNA in each instance. Moreover, it was observed that ETP-based isolation/purification of RNA yielded comparable or increased amounts of RNA as compared to the Promega-based column method (see FIG. 24).

### Example 13: Analysis of Nucleic Acids Obtained by ETP-based Isolation/Purification of FFPET Samples

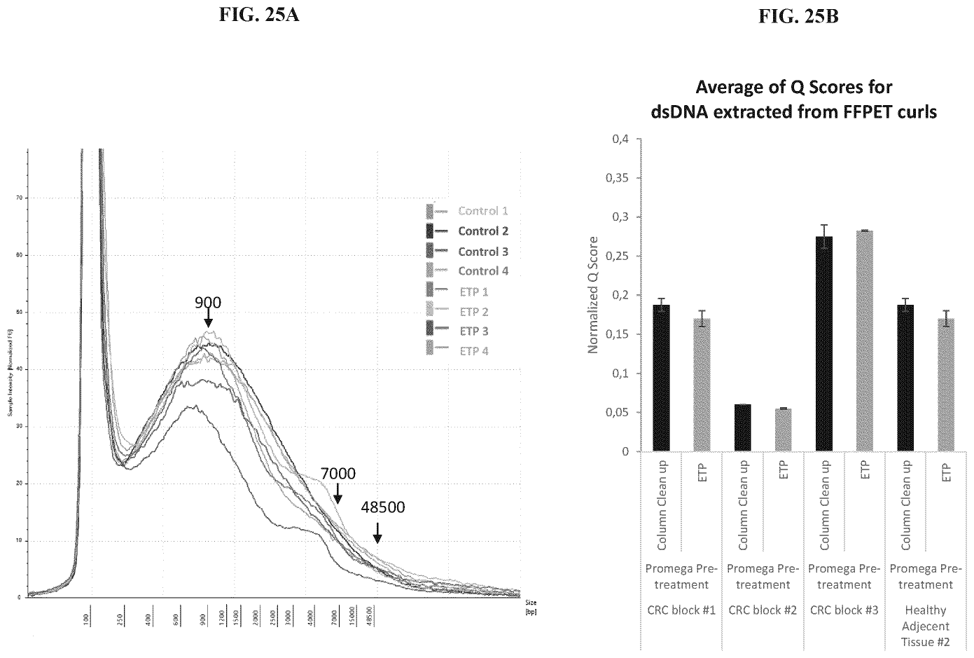

In the present example, nucleic acids obtained by ETP-based isolation/purification were analyzed by Agilent TapeStation based analysis to examine the size profile of the dsDNA isolated/purified. Furthermore, the dsDNA obtained by ETP-based isolation/purification was analyzed by quality control qPCR to obtain a Q score, a measure of nucleic acid quality as discussed above. Four ETP-based isolation/purification runs were performed to generate the data of FIG. 25A, and these data were compared to four controls. Additionally, four ETP-based runs with samples originating from CRC block 1, 2, 3, and healthy adjacent tissue #2, were performed and compared to the Q scores of dsDNA obtained using the Promega column-based method using the same four sample sources, i.e., CRC block 1, 2, 3, and healthy adjacent tissue #2 (FIG. 25B).

Referring now to FIG. 25A, it was observed that dsDNA obtained by ETP-based isolation/purification collected a range of DNA sizes, inclusive of relatively large dsDNA, as the predominant peak was at about 900 bp in size, with a further peak at approximately 7000 bp The results presented in FIG. 25A demonstrated the size profile of dsDNA obtained by the ETP-based isolation/purification was similar to that of the control.

Referring now to FIG. 25B, it was observed that dsDNA obtained by ETP-based isolation/purification had similar or improved Q score as compared to dsDNA obtained by the Promega column-based method thereby demonstrating that high quality dsDNA was obtained using the ETP-based isolation/purification.

### Example 14: Total Nucleic Acid Isolation/Purification from FFPET Samples by ETP and Nucleic Acid Yield Analysis

In the present example, ETP-based isolation/purification was used to isolate/purify the total nucleic acid content FFPET samples from two different CRC blocks, and the yield of dsDNA and RNA from said ETP-based isolation and purification was compared to the dsDNA and RNA obtained using the KAPA-beads based method as generally described in Example 8. dsDNA yield and RNA yield was quantified using a Qubit fluorimeter. 2 duplicates were performed of each run, and statistically significant results were those with P<0.05. Furthermore, it is noted that the KAPA Express Extract-based protocol, rather than the Promega-based protocol, was used prior to the ETP runs for FFPET sample preparation.

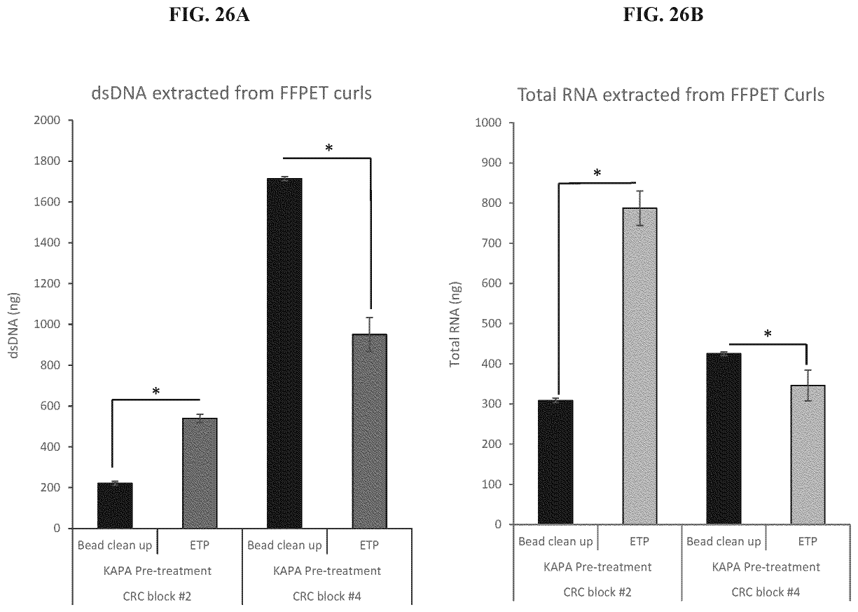

Referring now to FIG. 26A, ETP-based isolation/purification of dsDNA from CRC block 2 and CRC block 4 yielded desirable levels of dsDNA in each instance. Moreover, ETP-based isolation/purification of dsDNA resulted in a higher yield of dsDNA from CRC block 2 as compared to the KAPA-beads based method.

Referring now to FIG. 26B, ETP-based isolation/purification of RNA from CRC block 2 and CRC block 4 yielded desirable levels of RNA in each instance.

Moreover, ETP-based isolation/purification of dsDNA resulted in a higher yield of RNA from CRC block 2 as compared to the KAPA-beads based method.

### Example 15: Total Nucleic Acid Isolation/Purification from FFPET Samples by ETP and Nucleic Acid Analysis

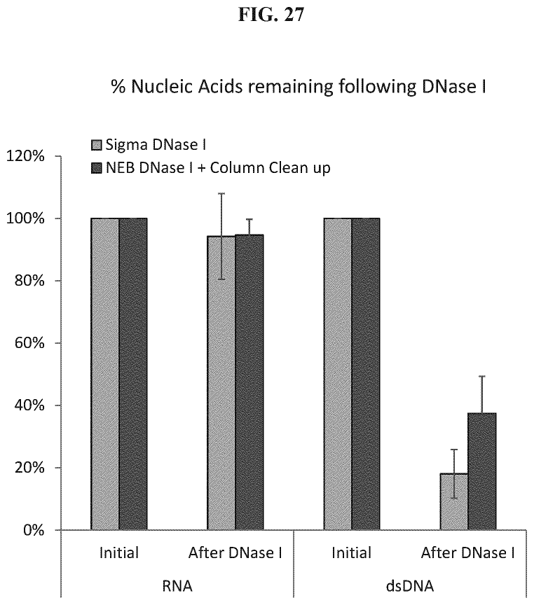

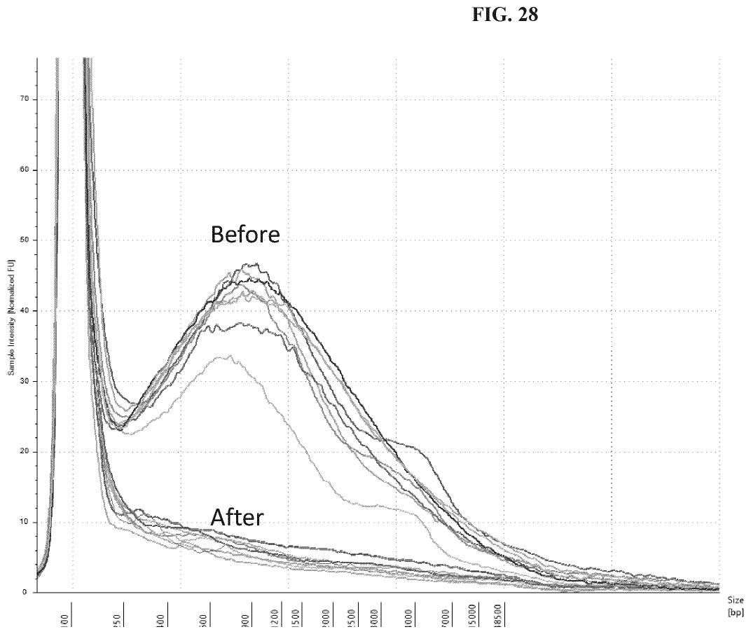

In the present example, ETP-based isolation/purification was used to isolate/purify nucleic acids from FFPET samples, and the collected nucleic acids were subjected to DNase I treatment. As presented in FIG. 27, two different DNase I treatments were performed on separate ETP isolated/purified samples: Sigma DNase I and NEB DNase I+column cleanup. Furthermore, four different nucleic acid samples collected after ETP-based isolation/purification were compared to four control samples both before and after DNase treatment of the ETP-based samples and the control samples by Agilent TapeStation-based analysis (see FIG. 28).

Referring now to FIG. 27, both DNase I treatments demonstrated degradation of DNA comprised by each of the samples, thereby validating the RNA results of the previous examples described supra.

Referring now to FIG. 28, isolated/purified nucleic acid samples (both control and ETP-based) demonstrated a change in size profile before and after DNase I treatment consistent with the DNase I treatment effectively degrading DNA contained by the samples, thereby further confirming the RNA results of the previous examples described supra.

### Example 16: Simultaneous Extraction of DNA and RNA from FFPET (Colorectal Cancer)

In this example, We extracted DNA and RNA from 5 CRC FFPET blocks using ETP as described herein and for comparison, the Promega FFPET DNA Column clean up (according to manufacturer's instructions, except the RNase step was excluded). The nucleic acid yield was evaluated using Qubit fluorometer (ThermoFisher Scientific, Carlsbad, Calif.). The DNA amount was measured following RNase treatment and DNA column clean up. The RNA amount was measured following DNase treatment and RNA column clean up. The quality of the nucleic acids was assessed for the ETP-isolated material and for DNA only by PCR.

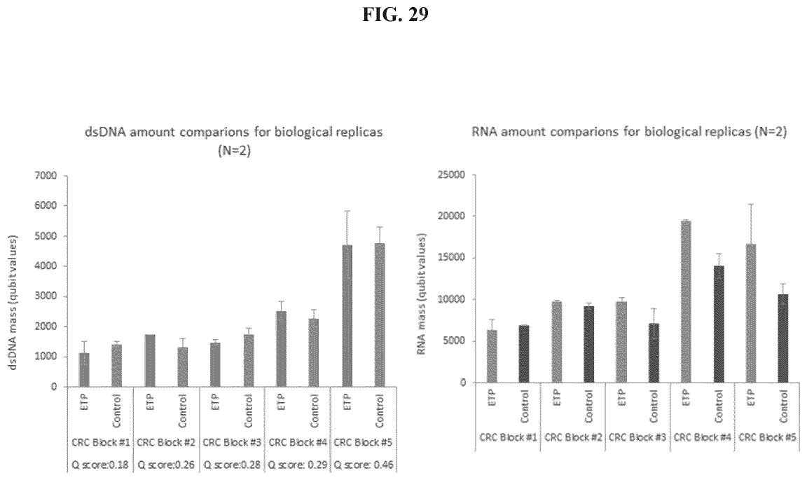

The quality is represented by the Q score (range 0-1). The results are shown in FIG. 29. The results demonstrate that the method and apparatus disclosed herein exceed the performance of the state-of-the-art method in 8/10 tests.

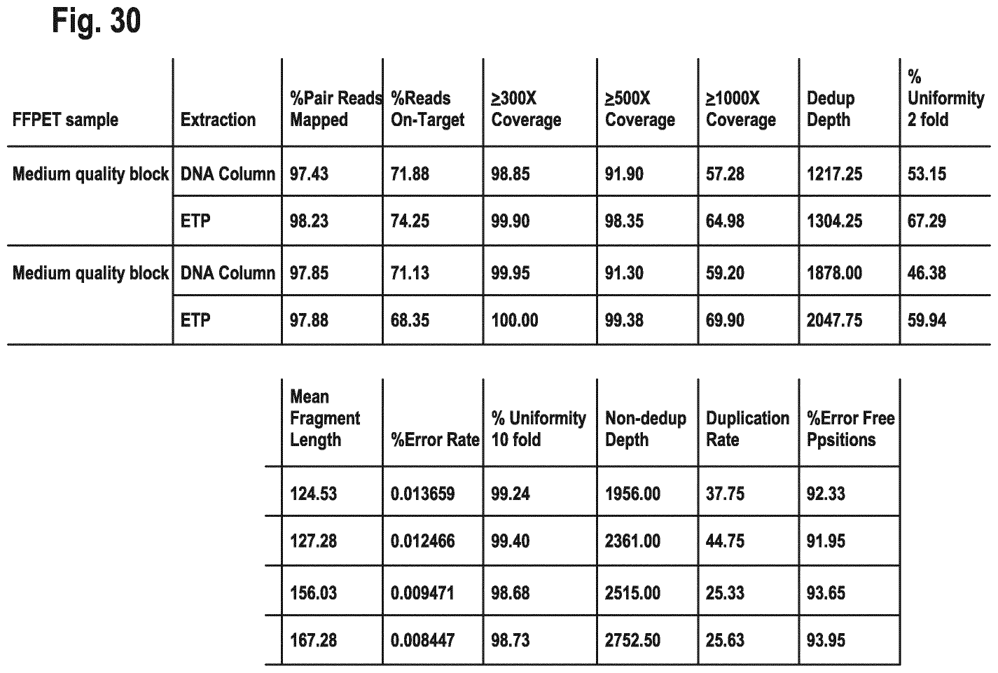

For each extraction method, two extraction replicas we subjected to nucleic acid sequencing on the Illumina platform. Two sequencing runs were performed for each replica (four sequencing runs for each method of extraction). AVENIO Library Prep kits (Roche Sequencing Solutions, Pleasanton, Calif) were used to prepare sequencing libraries and perform target enrichment. The sequencing results are shown in FIG. 30. The sequencing data obtained with the nucleic acids isolated as disclosed herein is superior in nearly every metric tested.

In the preceding procedures, various steps have been described. It will, however, be evident that various modifications and changes may be made thereto, and additional procedures may be implemented, without departing from the broader scope of the procedures as set forth in the claims that follow.

1. A method of isolating nucleic acids from a Formalin-Fixed Paraffin-Embedded Tissue (“FFPET”) sample, wherein said method comprises:
i. providing a device for effecting epitachophoresis (ETP) comprising a circular outer electrode encircling a gel comprising a leading electrolyte (LE), a central electrode and a collection well ;
ii. preparing from an FFPET sample an FFPET sample solution in a trailing electrolyte (TE);
iii. performing an epitachophoresis run by applying constant power to said device to focus the nucleic acids into one or more focused zones; and
iv. collecting said one or more focused zones from the collection well thereby obtaining a solution of isolated nucleic acids.

2. The method of claim 1, wherein preparing the FFPET sample comprises deparaffinization and lysis.

3. The method of claim 1, wherein the FFPET sample solution further comprises a dye and the method further comprises monitoring the migration of the dye and collecting the focused zones when the dye reaches the collection well.

4. The method of claim 1, wherein the nucleic acids are a combination of DNA and RNA.

5. The method of claim 1, wherein the isolated nucleic acid is of a predetermined size.

6. The method of claim 1, further comprising isolating DNA by treating the solution of isolated nucleic acids with RNase.

7. The method of claim 1, further comprising isolating RNA by treating the solution of isolated nucleic acids with DNase.

8. The method of claim 1, wherein the leading electrolyte comprises HCL-histidine.

9. The method of claim 1, wherein the trailing electrolyte comprises MES or TAPS.

10. A system for isolating nucleic acids from Formalin-Fixed Paraffin-Embedded Tissue (“FFPET”) samples, wherein the system comprises:
i. a device for effecting epitachophoresis (ETP) comprising a circular outer electrode encircling a gel comprising a leading electrolyte (LE), a sample loading area, a central electrode and a collection well;
ii. a power supply; and
iii. means for collecting a sample from the collection well.

11. The system of claim 10, further comprising a means of monitoring the migration of nucleic acids through the gel.

12. The system of claim 10, further comprising a means of coupling the monitoring with the collecting.

13. The system of claim 10, further comprising a means for loading a sample into the sample loading area.
